SEQUENCE LISTING (16 sequences)

SEQ ID NO: 1 - protein, 131 aa, Homo sapiens
  VARIANT (1)...(131): eppin-like precursor
MGLSGLLPILVPFILLGDIQEPGHAEGILGKPCPKIKVECEVEEIDQCTKPRDCPENMKCCPFSCGKKCLDFRKDICSMPQEAGPCLASIPHWWYNKKTKICSEFIYGGCQGNNNNFQTEAICLVTCKKYH

SEQ ID NO: 2 - protein, 106 aa, Homo sapiens
  VARIANT (1)...(106): mature form
EGILGKPCPKIKVECEVEEIDQCTKPRDCPENMKCCPFSCGKKCLDFRKDICSMPQEAGPCLASIPHWWYNKKTKICSEFIYGGCQGNNNNFQTEAICLVTCKKYH

SEQ ID NO: 3 - DNA, 11201 bp, Homo sapiens
  misc_feature (1)...(11201): public sequence AL031663.2 (94000..105200)
tttaaagaatcatgtaaaacaggcttacaaattttctttttaaaaatattttattttattttacgttccgggatacatgtgcaggacctgcaggtttgttacataggtaagcctgtgccatggtggtttgctgcacctatcaacccattacctgggtattaagccccgcatgcattagctatttatcctgatgctttccctccccctgccccctcaacagtccacgtgttctcattgttcatctcccacttgcaagtgagaacatgtggtatttacttttgttcctgcgttactttgctgaggataacggcttccagcttcatccatgtccctgcaaaggacgtgatctcatttctttttatggctgcatagtattccatggcatatatgtaccacattttctttatccagcctatcgttgatggatatttggtttgattccatgtttttgctattgtaaatagtgctgcagtgaacatacacatgcatgtatctttataaaagaatgatttatattcctttgagtgtatacccagtaatgggattgctgggtcaaatggtatttctggttctagatctttcaggaattggcacactctcttccacaatggttgaaccaatttacattcccaacagtgtaaaagcattcatgtttctccacagctttaccagcatctgttgtttctggactttttaataatcaccattctgactggcatgagatggtatctcattgtggttttgatttgcatttctctgatgatcactggtgttgagctttttttcatatgtttgttggccgcataaatgtcttcttttgagaagtgtctgttcatgtcctttgcccacttttcaatggggttgtttgttttttttcttgtaaatttgtttaagttccttgtagattctggatatcagaccacaaattttcttaatctgtctccagtgatgggaaagctaagctatttgtccactgattcccaacgcccactggatgccagggatactaaatccctcgctctttcaaactgttatacttgcaagctatacaagctccctgggggccagagaaaacccacagcagaaaattagggactgatgctagaggtaggaggctgtcaatgtgcattggctattattcggccataaaaaggaatgaacttctcactcaccactgatgacatgccttgctgcacacacaccctgctgccaccctgcagaaatgcttccattacccacagtcctttgccagatggaactgatgcccaggtaactggctcctcacctccttcgggcttataccaaggatataaaacttggtacagatgctcaagcgttaatgcttcagggtgtgacttttaatccaatgctgtagctgtgacaacagggaaaaacggagaacagagttggagaagtcccaaagtaatgaaagaagttatttactgttgcaaaatcaaacgacttaaaggataaatgtaactgatctttacatacgaaagaagagagagggaagtgaagggcggggagaaagagactcagacagacagagactccctgagaaaaagacaggcacagagtaaaagacatacagatgcacagaaaagagaagaaacagagtgagaaaaataaaacataagaatagacatgtcaggagaagagtgaaattagaaagaaggaagagagctagcatactgttaacctgcaaactccccctgggaaatggaagactctggagttaaacctccccttctgctaagggagcctggaaagcccaagacaacatgtgccctctttccctggtagaccagaaagcctcagattgctgaggattaggagccacctatcccccagtgtaggtaccaggtagtcccactgatggcctcagggatctggtgttcctgatcggggagtctggctggcttgtctccagggctgtctcttaaggtgctgggccacaccggggcaggcaaagtgcagatgtaggagctgctgaggagcagttcttagttttggtgccatcaaccaggccagtacttcctaaaatgggactctcaggacttctgccaatcctggtaccattcatccttttgggggacatccaggaacctgggcacgctgaaggcatccttggcagtaagtattgggaactccgtccttgcaatggggaaaggaaactcagtggggaggaaggaacaaagaataattttgcagctgagaaattattttctccacctctgcctggaattccaggtgggaaagaggggagctatttgaagtctcttggtaagagactcccttggtgaatacaggcatccaaatctgtggcaagtgactccttgctgccattccaaagagttcttcctggaggtgcctggacctagagctcatgaaatgaactaagaaggagtagtaagcccaaggaagtctcccacaagaaaaacaggctgctccccacaacaccacctgtgtcaacttccaaatgttctcattgcagcccccacacccatacccacagaggaccctggtctttgggttaaagaatggtctccagaagtcccttggaatgctgaaatgtgaagactcacaattagcagctaactgtactgggcactgaaaattagtggttgaaagtgagcactcaggagccacacattgtaggttcaaacctggctccacccctgagtagctgtgtgatcttgggcaaattcacataatctccttatgcttccaattcttctgtttacaaaatgtgaatattaagagtacttacccatagatttgtaatgaggattaaggagaaaaggcagctcacagtaagtgcttcatccatattactcattgttacgttacattgccccaggcagtttgacgatggcttgtcaaactgtgttgctggttagtcttcaaagcaaccctatgggcaaatgaggaaatcaaggctcagagagagtgcctggcacaccaagcgtctcacagaattcaaaagcccagctatctgatgccagaaatgttaatctcaatcatcgagagccacagcacctcctgggggagcaaccaagcacagcaaggctgagtgaccagaacagtcttagaggaatgcgtctttctctcaggaatctcatagatggtgcgggtagggggtaggggttggtttaggcatccgatggtgagttgttatggggttggggatgtgagcagtgttgtgaaattcctgagccccctgagggccactaccttctggggataggtgaagtggaaacaaagttctctaactctggaggggtgccctggtctcggggagggggagcactggctcctttaaggccttgagggaatatatcttcccctcccttagagccgtgtcccaaaatcaaagtggaatgcgaagtggaagaaatagaccagtgtaccaaacccagagattgcccagaaaacatgaagtgttgcccgttcagccgtggaaagaaatgtttagacttcagaaaggtaactcagatgcttcctaaattacccagtgccctcacctcctatctccacctgcactgcactacctctgtctcctagttcactgatggctggtctctgaccaagagtttaggtacatcttctacttgacacaaatctgccagtcctggacttgggagacatggattttatttcatttcttcctctattttggtaagtgatgtgaacaatgtagttttcctttctgggcctcagtttccctttccataagacaggtgtggataatggaccacatggtctcttggctactcttcagtgattctcgagatgccaagtagtcaccaaagtctcatcaaatgccaacattgtgtcaggttatatgatagctataagagacaggtatataatctgtatttcctggcttttttctctaggcctctctgtctacataaatccaggatgccttccctgaccaccttagaccgcctacactctttcttctctgcccatattattgagctctaaagaagatcatcaaggaccattcgttcattctattcattcaacaaatattgattggtgcccctgacacccatgatgggttccacttgctcattgtggccaggaaggtcagaaaagtttttctgggaaagagatccatgatttaagatctgaaagatgagtacttagaccaaaaatgggaagaaatagcctttcaggccagataataggatgtgcaaaggctctgtggcaggaggcaggatgttcaggtctagaaaggctagaaagcagagagtgagcaagagacaagaaacaggaaactggtagtgtatattcagggcaacttgaatctgtattcccaagtggccatcctcaagttttggggtcaaataaatgccctacttaatgatattttctgaatcttgttactttaggttgacatacctttcaagacaataactaggttttcagtgctttgataaaacctcaaatgtgtggccttccagccaggcatctgtggtttcatattgtttaagcatttgttgcatccctatggattgcagctctctataggttcgctgttgctgaaccaagaaataaggcatacacagatttctagagaaattgtaattctataacagattttcaggagatgaggaaggctggtatctttcagagtacaaagtgattttggaattgaaagaatttctttgggttaaagtttgtcactgacttgtgttcctgaaccatgaagcatgaatatgtgggctgagaaatagtttctcttgataaataaacaagtaacaaattttaaaaagagaggaagatgaagctgggctagttgggggaaggagagctggtaagggatctggtcttgatcctaacagcaatggattaacctatgttcaaatcccttaccatctgtgtggcctcaagaaagttatttagcctctctaaaccttagttttctcatctgcataatgagaacaatgagaggacctactttacaagattaaatgttaatagatgtcaactactaagaacactaatcaatgctataattgttgttttgttgtgctgacactaatcaatgctacaattgttgttttgtcgtgcttcagacctgaggttctaaagaactctatttgtacctcatagcactatgggcaaaataattgctcaataaatgcttaagatcagggtagatttgagttttgggtttttttttttttctgtgaatactcctgaagcatcctctctatgccaaattacctgctagaaactgctgtatcaagatggcataagatgcaatcccctctcaaaaacctcacattctcatggaggaatcagaaaatatgaacaagaaagtaaatggaaatgataaatgtgtgtgcaagactgagaagcaacgaagaaaagcaaagagtaaagaatgaatccaggaaagactgcataaaagagtcaatgtccggaaagggttttaaagaatgaacaagagttcacttagcaagagggggctggatatgtcaggcatgtgcaaaggcacggggtgtgtaagaaagtgctatttttagagcataagtttgggaagttgggtgttggggcagcataagaaataaagctggagaggagaaggagaaaatggattctgaagtccctctgggtttggtagaactttatcctgtgcccagcagatcggccattctggaaaccaatgttgagcagattggagaagtcttggagatcagctggtggcctttcatggagtccttgcaaaagataattatgcttgaattaagaaagcagccggaaggaaataaaggagagctatttaggaggcaaaatcagccagatttagtgttcagatgttggggaggcaaggaaaaagaagccatgtgatgtcacagctgctccttccaacagccctgttaggcgagcaggttgggattttgtattctcatcttacagatgaggaagtcaaggaccagagaggtaaaatgagtggtcagggttcacactgaattgggaacagagccctgaggcaaatttgcttctcagtggagacaaagggaatggtggccagagaaggggagagatggacattctccagggagacaggcacccaaacagctctattctgcgatctccctgcagatatatgcagtatgccacaggaggctggcccctgcctggcctccataccacactggtggtacaataaaaaaaactaagatctgctccgaattcatctatggcggtagccaggggaacaataacaacttccaaactgaagctatctgtctggtcacctgcaaaaaataccataagtcccagaggtcccggtctcctgtgctcaccaaggccacactgggaggtctgggtgttggctggtctattccaagacctgggtggcgctggggatgacaaaaccagctccaatgcagaagtataagtagaaggatattttgggaaagagggtgggaagggagggattagtcaaagggatattggcaagtatgaggtgagtagtgggtgtagagagaaaacagaagtggtggagtatcccagaccaggtcagacggaagcccggtaaacccagcccagccctgggcaccattcatcagccaatcattatagtcctttacttctcactaaaccttgttgctacttctcttcctttgaaaggttatttctaaccagggcaaccacatactttattggccaaaccaaatcacttttgaaagtgtctcaaggtgaggtgccattaattattacactgagacaacaggcataaactgggactctactggacaagtcagaactcatgatcattctaggagcccccaaactcaccttcattccattcctgcccaaagatgtaaaaatgatcccacctcctttttcccattaggtgcaagatttggttcctaatgtggtcagggtccaagcatctcaccctttattcttctgcttcacaggtcagccttactttataccataaggaggagcttgaataacctccaggatttggctcataatccaggcctctctccacgtgtgcctgattgatgctccaaattggcttccacgggccaaaccttggctgttccagaaactgaaccccaggaattgcttacacactttcttccagcgtagcatctcttcaaacacaatgctcttccccttgaccacttctcagtatgaaactctatgtcttcaactttcagaccccccatttatttgtatgaaggcttcagttgccttaataaagtgctctgtggggtccgatctctccgaggctgaataattcacatctgcaatatgctgatattgacatacactcacatctctctatatgcaattttcagagaacaaaaagccattgcattttctggtgacaccaaaaaagggagagagtatgaaaatcctgcaaattgagaatgctagtgacattgttactttggtaaagttgtcgttaagatgctgaagaatggagccagtgcagatccagattttgtatggtctaaacctgataaatgaggagctcctttagaaaccaagcacaggctgggcacggtggctcacgcctgtaatcccagcactttgggaggccaaggagggcggatcacctgaggtcaggagttcaagaccagcctgaccaacgtggagaaacaccatctctactaaaaatacaaaattagccaggcgtggtggtgcatgcctgtaatcccaactactcgggaggctgaggcaggagaattgtttgaacccaggaggcaaaagttgcagtgagctgagattgcaccattgcactccagcctgggcaataagaacaaaactcaaaaaaaaaaaaaaaaaaaaaagaaagaaagaaagaaaagaaaaaagaaagaaaccaagcacaaatgagtacaaaagagaatattatttcaaatgaggagggaaattgcaaccagctgcaaatttttaaaggctgacaaacagtaaaaattgcacaaaacccatataaattaataattcattgtttacatttatgacctcaattctgtaaagcactatttctgtggagagaatggaaagataactttttatttaatgtggggatcaaaatatgatattttaatttttattataggtgaattggataaaacatgtacctgaatgcttgaagccatgcttgatgtctgcagttctgttacaggtttgtgtcctagaaacacaggaattttaataaattatcttttgtacaactctattaaaaaaagaaaaaatgcatggtacatttaaaattgtacatattgaattatcaagtatatgcttactacaaaagaacttctgaacaggcttccataagagctgattttttttttctaattaaaatgtgatgtatctgaccagactcagtggttcatgcctgtaatcccagcactttgggaggccgaagggggcagatcacctgatgtcaagagttcaagaccagactggccaacgtggtgaaaccccatctctactaaaaatacaagaaattagccaggtgtggtggtgcacacttgtagccccagctactcaggaggctgaggcaggagaattgcttgaacccgggaggtggaggttgcagtgagccaagatcgtgccattgcactctagcctgggcggtggagtgagactccatctcaaaaaaaaaattgatgtatctgatatttgaaagaatgctacacaggcttgttttcctggctccacacatttcaaaacttgtttctcctctaccacccgcaaccttcttgggctaagaacattcattttacagtgcaacctctagtcccataacttagtgtcataacatcaagggaataggcccagtagaaaacaggagtattatttgcagctgcttctccaccaggacagctagcagcaacttcctacacatacaaatgacagctaacgacctaaatatattctactaactccaaaataaatatatccccaactcaacttccttttaatttgcatgcctaaaatgctggcagtcgcccaaacaccacctgacctaggagaagtgtgacagagggaaaggtcagagtggaaagggaaggtggattggcgattatggttagaatatcttacttttgcagatattacaagtcatctgaccatgtaaacatatagctagaagcactcccagaatcttaaaaggggctcgctcgagcgaggaccctgaggcttggctccatcgacctcatggtaagtccgtctctgaaagggctcatggacttgtttactggagttgtagtaaagaagacagttccatagtatggtgcagaaactccaaaattgaaaatagctggacaggcatgaaagggggagatggaggaaagcagagaagagttaaagaggaagggagaagaaaggggtgaaggaggaaaagacactggatggtgtgactgttttaaagcaaggtcctaaaattctttcactcaccttccacctagaagtgggatctatgtcccctccttttgaatctgagaaaactctgtgactgctttgaccaagagaataaagcaaaaatgatgctatgtgacctccaaggctaagctagaataagtgcaagtcccacttgatcctcttatgtagcttgtttgggggcagtgggaaactgccatgtaagatgtccaactactctgagagaggcccatgctagagagacccatgtgggtgctgtgatcagcaggcccagataagctctgactgatagccagcatcaatttcagccatgcaaatgagatgcccaacccaatagagccttcagatggctgcagccccagcagacatctgactgcaaccatgggaaagaccctataaaagaaccaccaagcagattgcttcccagatggctgacccacaatataaaatggttgctttaaagtgctaagttttcagaaaatttattcacagaataggtgatcaaagatggaaacgggctaggagcagtggctcacagctgtaatcccaggcactttgggagtccaaggcaggcagataacttgaagtcagaagttcaagaccagcctggccaaaacggcaaaaccctgtctctactaaaaatacaaaaattagccagacatagtggcgggcacctgtaataccacctactcgggaggctggggcaggagaatctcttcagcccgggaggcggaggttgcagtgagctgagattgcgccactgcactccagagcgagatttggtctcaaaaaaaaaaaaagaaagaaaaaaaagatgaaaagggagtagaaggaaggatagggagagagaaagaagaaagagacacccataggactgggcaagataagaggatgccaggcttgagaagggaacctcacaacaggcagcttgtcatcacagcacaccaagacttgctatctcttgtcaaataagtttgttgtttaatataacttggatcaaggggctctaaagaggctggaattcatttgctgttgatgctgttgccatctctgcttccctctctttagaaagggctggtaaaactgattgtagaaaactctaaaggcactgcaaaaaaaggttagaattaataaacaaatttattaaagttacaggatacaaaatcaacatacaaaaatcaatagcaactttatacaccaagaacaatctcatttccatcagcggcaacaacaacaaaacactcagtaattaactcaaccaaagaagtgaaaaacttatgcattgacaattataaaacatcgatgaaggaaatttaaaagacacaaataaatgaaaagacatccatgctcatagattagaagaatcaatgttgtcaaaatgtccacactacccaaagcaatctacagagcacatgcaatttctgccaaaatgccaatggcattttttacagaaacataaaaagcaatgctaaaattcatatggaaccacaaaagaccccaaattgccaaaggaattttgaaaaagaacagagctgtaagcaccacatttcctgatgtcaaattacattgcaaagctagagtaatcaaaagagtgtggtactggcataaaaacagaaaagcagacatacaggccaatggaacagaatagagaagccagaactaagcccacccatttatggtcaattgatctttgacgagggtgtcaaaaacacacaataggcaacagaagatctctttaacaaatggtgctgggaaaactagatattcacatgcaa

SEQ ID NO: 4 - protein, 30 aa, Homo sapiens
MGLSGLLPILVPFILLGDIQEPGHAEGILG

SEQ ID NO: 5 - protein, 43 aa, Homo sapiens
PCPKIKVECEVEEIDQCTKPRDCPENMKCCPFSCGKKCLDFRK

SEQ ID NO: 6 - protein, 24 aa, Homo sapiens
DICSMPQEAGPCLASIPHWWYNKK

SEQ ID NO: 7 - protein, 33 aa, Homo sapiens
TKICSEFIYGGSQGNNNNFQTEAICLVTCKKYH

SEQ ID NO: 8 - DNA, 1339 bp, Homo sapiens
  misc_feature (1)...(1339): reconstructed cDNA from SEQ ID NO3
atgggactctcaggacttctgccaatcctggtaccattcatccttttgggggacatccaggaacctgggcacgctgaaggcatccttggcaagccgtgtcccaaaatcaaagtggaatgcgaagtggaagaaatagaccagtgtaccaaacccagagattgcccagaaaacatgaagtgttgcccgttcagccgtggaaagaaatgtttagacttcagaaaggatatatgcagtatgccacaggaggctggcccctgcctggcctccataccacactggtggtacaataaaaaaaactaagatctgctccgaattcatctatggcggtagccaggggaacaataacaacttccaaactgaagctatctgtctggtcacctgcaaaaaataccataagtcccagaggtcccggtctcctgtgctcaccaaggccacactgggaggtctgggtgttggctggtctattccaagacctgggtggcgctggggatgacaaaaccagctccaatgcagaagtataagtagaaggatattttgggaaagagggtgggaagggagggattagtcaaagggatattggcaagtatgaggtgagtagtgggtgtagagagaaaacagaagtggtggagtatcccagaccaggtcagacggaagcccggtaaacccagcccagccctgggcaccattcatcagccaatcattatagtcctttacttctcactaaaccttgttgctacttctcttcctttgaaaggttatttctaaccagggcaaccacatactttattggccaaaccaaatcacttttgaaagtgtctcaaggtgaggtgccattaattattacactgagacaacaggcataaactgggactctactggacaagtcagaactcatgatcattctaggagcccccaaactcaccttcattccattcctgcccaaagatgtaaaaatgatcccacctcctttttcccattaggtgcaagatttggttcctaatgtggtcagggtccaagcatctcaccctttattcttctgcttcacaggtcagccttactttataccataaggaggagcttgaataacctccaggatttggctcataatccaggcctctctccacgtgtgcctgattgatgctccaaattggcttccacgggccaaaccttggctgttccagaaactgaaccccaggaattgcttacacactttcttccagcgtagcatctcttcaaacacaatgctcttccccttgaccacttctcagtatgaaactctatgtcttcaactttcagaccccccatttatttgtatgaaggcttcagttgccttaataaa

SEQ ID NO: 9 - protein, 98 aa, Homo sapiens
MGLSGLLPILVPFILLGDIQEPGHAEGILGKPCPKIKVECEVEEIDQCTKPRDCPENMKCCPFSRGKKCLDFRKDICSMPQEAGPCLASIPHWWYNKK

SEQ ID NO: 10 - protein, 33 aa, Homo sapiens; identical to SEQ ID NO: 7

SEQ ID NO: 11 - DNA, 396 bp, Homo sapiens
  misc_feature (1)...(396): reconstructed cDNA for SEQ ID NO1
atgggactctcaggacttctgccaatcctggtaccattcatccttttgggggacatccaggaacctgggcacgctgaaggcatccttggcaagccgtgtcccaaaatcaaagtggaatgcgaagtggaagaaatagaccagtgtaccaaacccagagattgcccagaaaacatgaagtgttgcccgttcagccgtggaaagaaatgtttagacttcagaaaggatatatgcagtatgccacaggaggctggcccctgcctggcctccataccacactggtggtacaataaaaaaactaagatctgctccgaattcatctatggcggttgccaggggaacaataacaacttccaaactgaagctatctgtctggtcacctgcaaaaaataccattaa

SEQ ID NO: 12 - protein, 131 aa, Homo sapiens; identical to SEQ ID NO: 1

SEQ ID NO: 13 - protein, 133 aa, Homo sapiens
MGSSGLLSLLVLFVLLANVQGPGLTDWLFPRRCPKIREECEFQERDVCTKDRQCQDNKKCCVFSCGKKCLDLKQDVCEMPKETGPCLAYFLHWWYDKKDNTCSMFVYGGCQGNNNNFQSKANCLNTCKNKRFP

SEQ ID NO: 14 - protein, 134 aa, Mus musculus
MKLSGFVSILVLFGLLARVQGPSLADLLFPRRCPRFREECEHQERDLCTRDRDCPKKEKCCVFNCGKKCLNPQQDICSLPKDSGYCMAYFRRWWFNKENSTCQVFIYGGCQGNNNNFQSQSICQNACEKKSSLT

SEQ ID NO: 15 - protein, 131 aa, Homo sapiens
MGLSGLLPILVPFILLGDIQEPGHAEGILGKPCPKIKVECEVEEIDQCTKPRDCPENMKCCPFSRGKKCLDFRKDICSMPQEAGPCLASIPHWWYNKKTKICSEFIYGGSQGNNNNFQTEAICLVTCKKYH

SEQ ID NO: 16 - protein, 136 aa, Mus musculus
MRLWGLLPFLVPFILLWSIQEPELAEGFFIRTCPRVRVKCEVEERNECTRHRQCPNKKRCCLFSCGKKCMDLRQDVCSLPQDPGPCLAYLPRWWYNQETDLCTEFIYGGCQGNPNNFPSEGICTVVCKKKQMSSWI

(19) United States
(12) Patent Application Publication
(10) Pub. No.: US 2004/0229312 A1
(43) Pub. Date: Nov. 18, 2004

(54) Engineered human kunitz-type protease inhibitor

(76) Inventors: Lydie Bougueleret, Petit-Lancy (CH); Amos Bairoch, Vessy (CH); Anne Niknejad, Bellevue (CH)

Correspondence Address: PERKINS COIE LLP, P.O. BOX 2168, MENLO PARK, CA, 94026, US

(21) Appl. No.: 10/807,204

(22) Filed: Mar. 22, 2004

Related U.S. Application Data
(63) Continuation of application No. PCT/EP03/01629, filed on Feb. 18, 2003.
(60) Provisional application No. 60/358,683, filed on Feb. 21, 2002.

(51) Int. Cl. C07H 21/04; C07K 14/705

(52) U.S. Cl. 435/69.1; 435/320.1; 435/325; 530/350; 536/23.5

(57) ABSTRACT

The invention provides a novel human DJ11 Kunitz-type protease inhibitor having an amino acid sequence of SEQ ID NOS 1 or 2. The invention provides compositions including polypeptides, polynucleotides, and antibodies specific for the human DJ11. Also provided are methods for making such compositions, and methods of using the compositions of the invention for diagnosis, prognosis, and treatment of diseases.

### FIELD OF THE INVENTION

The present invention relates to an isolated human DJ11 and its precursors, isolated polynucleotides encoding such polypeptides, polymorphic variants thereof, and compositions for detecting and using such polynucleotides and polypeptides.

### BACKGROUND

In animals, proteinases are important in wound healing, extracellular matrix destruction, tissue reorganization, and in cascades leading to blood coagulation, fibrinolysis, and complement activation. Proteinases are released by inflammatory cells for destruction of pathogens or foreign materials, and by normal and cancerous cells as they move through their surroundings. One family of proteinase inhibitors, the Kunitz inhibitors, includes inhibitors of trypsin, chymotrypsin, elastase, kallikrein, plasmin, coagulation factors XIa and IXa, and cathepsin G. These inhibitors thus regulate a variety of physiological processes, including blood coagulation, fibrinolysis, and inflammation.

Proteinase inhibitors regulate the proteolytic activity of target proteinases by occupying the active site and thereby preventing occupation by normal substrates. Although proteinase inhibitors fall into several unrelated structural classes, they all possess an exposed loop (variously termed an “inhibitor loop”, a “reactive core”, a “reactive site”, or a “binding loop”) which is stabilized by intermolecular interactions between residues flanking the binding loop and the protein core (Bode and Huber, Eur. J. Biochem. 204:433-451, 1992). Interaction between inhibitor and enzyme produces a stable complex which disassociates very slowly, releasing either virgin (uncleaved) inhibitor, or a modified inhibitor that is cleaved at the scissile bond of the binding loop.

The Kunitz inhibitors are generally basic, low molecular weight proteins comprising one or more inhibitory domains (“Kunitz domains”). The Kunitz domain is a folding domain of approximately 50-60 residues which forms a central anti-parallel beta sheet and a short C-terminal helix. This characteristic domain comprises six cysteine residues that form three disulfide bonds, resulting in a double-loop structure. Between the N-terminal region and the first beta strand resides the active inhibitory binding loop. This binding loop is disulfide bonded through the P2 Cys residue to the hairpin loop formed between the last two beta strands. Isolated Kunitz domains from a variety of proteinase inhibitors have been shown to have inhibitory activity (e.g., Petersen et al., Eur. J. Biochem. 125:310-316, 1996; Wagner et al., Biochem. Biophys. Res. Comm. 186:1138-1145, 1992; Dennis et al., J. Biol. Chem. 270:25411-25417, 1995).

Kunitz-domain inhibitors are slow, tight-binding, reversible inhibitors of serine proteases that bind to the active site and inhibit according to the standard mechanism. Subsequent cleavage between the P1 and P1′ residues occurs very slowly if at all (Bode, W. and Huber, R., Eur. J. Biochem. 204: 433-451, 1992; Laskowski, M., Jr. and Kato, I., Annu. Rev. Biochem. 49: 593-626, 1980). There are many interactions between the serine protease subsites and the side chains in the primary binding loop of Kunitz domains (P5-P4′); however, the interactions of the P1 residue with the specificity pocket are energetically most important and therefore represent the primary specificity determinants.

Proteinase inhibitors comprising one or more Kunitz domains include bovine pancreatic trypsin inhibitor (BPTI), tissue factor pathway inhibitor (TFPI), tissue factor pathway inhibitor 2 (TFPI-2), amyloid beta-protein precursor (AβPP), and placental bikunin. TFPI, an extrinsic pathway inhibitor and a natural anticoagulant, contains three tandemly linked Kunitz inhibitor domains. The amino-terminal Kunitz domain inhibits factor VIIa (TF-FVIIa), plasmin, and cathepsin G; the second domain inhibits factor Xa, trypsin, and chymotrypsin; and the third domain has no known activity (Petersen et al, supra). TFPI-2 has been shown to be an inhibitor of the amidolytic and proteolytic activities of TF-FVIIa, factor XIa, plasma kallikrein, and plasmin (Sprecher et al., Proc. Natl. Acad. Sci. USA 91:3353-3357, 1994; Petersen et al., Biochem. 35:266-272, 1996). The ability of TFPI-2 to inhibit TF-FVIIa and its relatively high levels of transcription in umbilical vein endothelial cells, placenta and liver suggests a specialized role for this protein in hemostasis (Sprecher et al., ibid.). Aprotinin (bovine pancreatic trypsin inhibitor) is a broad spectrum Kunitz-type serine proteinase inhibitor that has been shown to prevent activation of the clotting cascade. Aprotinin is a moderate inhibitor of plasma kallikrein and plasmin, and blockage of fibrinolysis and extracorporeal coagulation have been detected in patients given aprotinin during open heart surgery (Davis and Whittington, Drugs 49:954-983, 1995; Dietrich et al., Thorac. Cardiovasc. Surg. 37:92-98, 1989). Aprotinin has also been used in the treatment of septic shock, adult respiratory distress syndrome, acute pancreatitis, hemorrhagic shock, and other conditions (Westaby, Ann. Thorac. Surg. 55:1033-1041, 1993; Wachtfogel et al., J. Thorac. Cardiovasc. Surg. 106: 1-10, 1993). Placental bikunin is a serine proteinase inhibitor containing two Kunitz domains (Delaria et al., J. Biol. Chem. 272:12209-12214, 1997). Individual Kunitz domains of bikunin have been expressed and shown to be potent inhibitors of trypsin, chymotrypsin, plasmin, factor XIa, and tissue and plasma kallikrein (Delaria et al, supra).

Substrates and inhibitors of TF-FVIIa and other trypsin-like proteases such as factor XIa and kallikrein have either Arg or Lys at the P1 residue. Therefore, at position P1 (amino acid 15 of aprotinin), either Arg or Lys is generally preferred. However methionine is sometimes found at the P1 position and may also be preferable for inhibition of some serine proteases (McGrath, M. E. et al., J. Biol. Chem. 266:6620-6625, 1991). The introduction of residues such as Val, Leu, or Ile at the P1 position of aprotinin may lead to potent inhibitors with differing specificity, e.g. to human polymorphonuclear leukocyte elastase (Beckmann, J. et al., Eur. J. Biochem. 176: 675-682, 1988; Sinha, S. et al., J. Biol. Chem. 266: 21011-21013, 1991). Residues other than naturally occurring amino acids have also been substituted into Kunitz domains and other related protease inhibitor domains by chemical synthesis (Beckmann, J. et al., Eur. J. Biochem. 176: 675-682, 1988; Bigler, T. L. et al., Prot. Sci. 2: 786-799, 1993).

Inflammatory Diseases

The inflammatory response after surgeries, trauma and infection involves neutrophil activation and infiltration into the injured tissue. The activated neutrophils release the neutral serine proteinases leukocyte elastase, cathepsin G and proteinase 3, which, if not properly controlled, cause abnormal connective tissue turnover and result in severe damage to healthy tissue (1-Watorek et al., Adv Exp Med Biol 1988;240:23-31; Murata et al., Inflammation, 1994;18(4):337-47; Yavin and Fridkin, J Pept Res, 1998;51(4):282-9). The uncontrolled proteolysis can lead to a myriad of diseases including emphysema, idiopathic pulmonary fibrosis, adult respiratory distress syndrome, cystic fibrosis, rheumatoid arthritis, organ failure, and glomerulonephritis.

Inhibitors of neutral serine proteinases released by neutrophils can have therapeutic efficacy in treating inflammatory diseases. In patients suffering from hyperdynamic septic shock, plasma levels of the serine proteinase inhibitors antithrombin III, alpha 2-macroglobulin and inter-alpha-trypsin inhibitor, as well as those of various clotting, complement and other plasma factors, are significantly decreased (Fritz, H., Ciba Found Syrnp 1979;(75):351-79). In an experimental endotoxenia model, the reduction in the plasma levels of these factors was considerably diminished by the intravenous injection of a soybean-derived leukocyte elastase and cathepsin G inhibitor, indicating that these neutral proteinases are at least partially responsible for the proteolysis of the plasma factors. In addition, the survival rate in the rat lethal peritonitis model (cecal ligation and puncture-induced septic shock model) was improved by treatment with the second domain of human urinary trypsin inhibitor (2), which has been shown to inhibit leukocyte elastase and cathepsin G (Swaim and Pizzo. Biochem J, 1988;254(1): 171-8; Morishita et al., Thromb Res, 1994;73(3-4): 193-204).

Stimulated neutrophils generate active oxygen species which contribute to inflammatory diseases, necrosis of surrounding tissues, mutagenicity and carcinogenicity (Frenkel, K., et al., Carcinogenesis 1987;8(9):1207-12). The most effective serine protease inhibitors in decreasing H₂O₂ formation by TPA-activated neutrophils were chymotrypsin-specific inhibitors (e.g., potato inhibitor-1 and a chymotrypsin-inhibitory fragment of potato inhibitor-2), followed by bifunctional inhibitors recognizing both chymotrypsin and trypsin, and least active was soybean trypsin inhibitor, a predominantly trypsin inhibitor. In addition, cytin, a chymotrypsin- but not trypsin-specific inhibitor, significantly diminished the level of human neutrophil and monocyte activation induced by lipopolysaccharide (Chopin et al., Eur J Biochem, 1997;249(3):733-8).

Neutrophil chemotaxis also plays an important role in the inflammatory response and, when excessive or persistent, may augment tissue damage (Lomas et al., J Biol Chem, 1995;270(40):23437-43). Inhibitors of cathepsin G and chymotrypsin suppressed neutrophil chemotaxis to the chemoattractants N-formyl-L-methionyl-L-leucyl-L-phenylalanine (fMLP) and zymosan-activated serum in multiple blind well assays and to fMLP in migration assays under agarose.

IL-1, a proinflammatory cytokine, is secreted from monocytes at inflammatory sites as an inactive precursor. Leukocyte elastase and cathepsin G cleave the IL-1 precursor to form fully active forms of IL-1 (Hazuda et al., J Biol Chem, 1990;265(11):6318-22). Synovial fluid collected from patients with inflammatory polyarthritis and bronchoalveolar lavage fluid from patients with sarcoidosis process the IL-1 precursor into the same active forms as leukocyte elastase and cathepsin G. Control fluids from patients who had no symptoms of inflammatory disease did not exhibit the processing activity. Only lavage fluids that processed precursor IL-1 contain cathepsin G and/or elastase activity.

Synthetic tannin exhibits anti-inflammatory properties in skin diseases. Tannin specifically inhibits leukocyte elastase in an irreversible manner, and it is believed that the anti-inflammatory properties of synthetic tannin may at least in part be due to inactivation of elastase (Mrowietz et al., J Invest Dermatol,1991;97(3):529-33).

Lung Injury

Many syndromes of lung injury, including emphysema, adult respiratory distress syndrome, cystic fibrosis and idiopathic pulmonary fibrosis, are associated with accumulation of neutrophils within the pulmonary parenchyma. Activated neutrophils have the capacity to produce lung injury by secreting products including proteinases and reactive oxygen species (Palmgren et al., J Allergy Clin Immunol, 1992;89(4):905-15). Neutral serine proteinases secreted from activated neutrophils are capable of inducing damage to lung alveolar extracellular matrix (ECM) by directly digesting the matrix and through the activation of latent metalloproteases resident in the matrix (Ferry et al., FEBS Lett, 1997;402(2-3):111-5). Proteinase 3 and leukocyte elastase have been shown to cause significant lung damage and emphysema when administered by tracheal insufflation or injection to hamsters (Kao, R. C., et al., (1988) J. Clin. Invest. 82, 1963-1973; Senior, R. M., et al., Am Rev Respir Dis, 1977;1 16(3):469-75).

Inhibitors of neutrophil neutral serine proteinases have been shown to exert potent therapeutic effects on pulmonary emphysema, adult respiratory distress syndrome and other diseases involving tissue degradation. Treatment of hamsters with Eglin C, a neutral serine proteinase inhibitor, completely protected hamsters against leukocyte elastase-induced emphysema (Schnebli et al., Eur J Respir Dis Suppl 1985;139:66-70). Derivatives of 5-methyl-4H-3,1-benzoxazin-4-one, shown to be highly specific inhibitors of leukocyte elastase, efficiently prevented degradation of insoluble elastin by stimulated neutrophils (Uejima et al., J Pharmacol Exp Ther, 1993;265(2):516-23). These small molecule inhibitors also significantly suppressed leukocyte induced pulmonary hemorrhage and emphysema in hamsters. Alpha 1-proteinase inhibitor and soybean trypsin inhibitor, two leukocyte elastase and cathepsin G inhibitors, were also shown to completely or nearly completely inhibit neutrophil-induced ECM solubilization.

Vascular Effects

Injury to the vascular endothelium, such as that occurs during angioplasty, can result in the accumulation of neutrophils and platelets and platelet activation at the site of injury. Platelet accumulation and activation at the injured site can result in abrupt artery closure. Cathepsin G potently induces platelet aggregation, secretion and calcium mobilization by binding to a specific receptor on platelets (Selak M A. Thromb Haemost, 1992;68(5):570-6). Leukocyte elastase, though having no platelet agonist activity itself, increases the apparent affinity of cathepsin G binding to platelets and enhances cathepsin G-induced platelet activation. Throinbospondin 1, which inactivates cathepsin G by binding near the enzyme's active site, protects fibronectin from cleavage by cathepsin G and blocks cathepsin G-mediated platelet aggregation (Hogg et al., J Biol Chem, 1993;268(29):21811-8).

Endothelin-1 (ET-1) is a potent vasoconstrictor peptide secreted by endothelial cells. Marked ET-1 degradation is observed in the presence of activated neutrophils. ET-1 inactivation could play a role in acute inflammatory reactions where neutrophils adhere to the vascular endothelial cells. Soybean trypsin inhibitor abolishes ET-1 degradation almost completely, suggesting a role of cathepsin G in ET-1 hydrolysis (Fagny et al., Regul Pept, 1992;42(1-2):27-37). Among the purified leukocyte enzymes tested, cathepsin G hydrolyzed ET-1 at the highest rate.

Cathepsin G converts angiotensinogen and angiotensin I to angiotensin II (Wintroub et al., Biochemistry, 1984;23(2):227-32; Tonnesen et al., J Clin Invest 1982, 69(1):25-30). The neutrophil-angiotensin system does not require renin or converting enzyme and may function as a mobile effector pathway which modulates tissue blood flow and/or vascular permeability.

Hemostasis and Blood Clotting

Hemostatic conditions including acquired coagulopathies can result from liver disease, uremia, acute disseminated intravascular coagulation, post-cardiopulmonary bypass, massive transfusion, or Warfarin overdose (Humphries, Transfusion Medicine 1:1181-1201, 1994). A deficiency or dysfunction in any of the procoagulant mechanisms predisposes the patient to either spontaneous hemorrhage or excess blood loss associated with trauma or surgery. Acquired coagulopathies usually involve a combination of deficiencies, such as deficiencies of a plurality of coagulation factors, and/or platelet dysfunction. In addition, patients with liver disease commonly experience increased fibrinolysis due to an inability to maintain normal levels of α2-antiplasmin and/or decreased hepatic clearance of plasminogen activators (Shuman, Hemorrhagic Disorders, in Bennet and Plum, eds. Cecil Textbook of Medicine, 20th ed., W. B. Saunders Co., 1996). Primary fibrinolysis results from a massive release of plasminogen activator. Conditions associated with primary fibrinolysis include carcinoma of the prostate, acute promyelocytic leukemia, hemangiomas, and sustained release of plasminogen activator by endothelial cells due to injection of venoms. The condition becomes critical when enough plasmin is activated to deplete the circulating level of α2-antiplasmin (Shuman, 1996). Data suggest that plasmin on endothelial cells may be related to the pathophysiology of bleeding or rethrombosis observed in patients undergoing high-dose thrombolytic therapy for thrombosis. Plasmin may cause further damage to the thrombogenic surface of blood vessels after thrombolysis, which may result in rethrombosis (Okajima, J. Lab. Clin. Med. 126:1377-1384, 1995).

Proteolysis of extracellular matrix, connective tissue, and other tissues and organs is an element of many diseases. This tissue destruction is believed to be initiated when plasmin activates one or more matrix metalloproteinases (e.g., collagenase and metalloelastases). Matrix metalloproteinases (MMPs) are believed to play a role in metastases of cancers, abdominal aortic aneurysm, multiple sclerosis, rheumatoid arthritis, osteoarthritis, trauma and hemorrhagic shock, and cornial ulcers. Administration of an MMP inhibitor, or a plasmin inhibitor, after hemorrhage improves cardiovascular response, hepatocellular function, and microvascular blood flow in various organs (Wang, Shock 6:377-382, 1996).

Kunitz domain proteins have been applied as therapeutics in a number of disease indications, as described above (see also, Greenwood, J., 1991, Neuroradiology 33:95-100; Coleman, 1984, J. Clin. Invest. 73:1249; Vallet et al., Nature 389:607; Chraibi et al., J. Gen. Physiol., 111:127; Huang, Y. et al., 1998, J. Leukoc. Biol. 64: 322-30; Dela Cadena, R. et al., 1995, FASEB J. 9:446-452; Laurenti et al., 1996, Diabetic Medicine 13:642-645). However, known Kunitz-type inhibitors may lack specificity and have low potency. Lack of specificity can result in undesirable side effects, such as nephrotoxicity that occurs after repeated injections of high doses of aprotinin. These limitations may be overcome by preparing isolated Kunitz domains, which may have fewer side effects than traditional Kunitz-type inhibitors used as anticoagulants. In addition, alpha 1-proteinase inhibitor, the major endogenous serine proteinase inhibitor for neutrophil elastase, is easily inactivated by proteolysis by metalloproteinases present in the injured lung and by oxidation, resulting in the pathogenesis of pulmonary emphasema (Doring G. Am J Respir Crit Care Med, 1994;150(6 Pt 2):S114-7; Smith et al., FEBS Lett, 1996;390(2):187-90; Jallat et al., Rev Fr Transfus Immunohematol,1986;29(4):287-98). Thus, an alternative inhibitor of neutral serine proteases is desired as a treatment for disorders such as pulmonary emphysema, arthritis, and other disorders related to inflammation and fibroblast proliferation (e.g., scarring). As such, there is a need in the art for additional Kunitz-type proteinase inhibitors exhibiting advantageous substrate and stability profiles.

### SUMMARY OF THE INVENTION

It is an object of the present invention to provide novel serine protease inhibitor proteins. The invention provides a pseudogene for a novel Kunitz-type inhibitor, as well as customized or ‘engineered’ active Kunitz-type serine protease inhibitors (i.e., proteins not normally present in nature). It is another object of the invention to provide materials and methods for making the protease inhibitor proteins. It is a further object of the invention to provide antibodies that specifically bind to the protease inhibitor proteins. The present invention is directed to compositions and uses related to the DJ11 gene, cDNA and engineered variants thereof. Such compositions include DJ11 polypeptides, DJ11 signal peptides, mature DJ11 polypeptides, fragments of DJ11 polypeptides comprising domains such as a Kunitz-type domain and/or a WAP domain, DJ11 antibodies, including monoclonal antibodies and other binding compositions derived therefrom, and methods of making and using these compositions.

In another aspect, the invention includes polynucleotides coding for a polypeptide having an amino acid sequence selected from the group consisting of SEQ ID NOS 1 and 2, antisense oligonucleotides complementary to such sequences, oligonucleotides complementary to DJ11 gene sequences useful in diagnostic and analytical assays, such as primers for polymerase chain reactions (PCRs), and vectors for expressing DJ11 polypeptides. The invention further includes methods of using DJ11 compositions, including antisense and antibody compounds, for example to detect DJ11 or assess function of a DJ11 of SEQ ID NOS 1 or 2 in a cell or animal, and methods of using DJ11 compositions, including primers complementary to the DJ11 gene and/or messenger RNA and anti-DJ11 antibodies, for detecting and measuring quantities of the DJ11 transcripts of SEQ ID NO: 4 or 5 in tissues and biological fluids. Such methods may be useful for determining levels of translationally inactive DJ11 in an animal, preferably a mammal such as a human.

In another aspect, the invention includes an isolated polynucleotide having a sequence at least 95 percent identical to any sequence of 100 to 1000 consecutive nucleotides selected from the group consisting of SEQ ID NOs: 3-5, or any complementary sequence of the foregoing. More preferably, the invention includes an isolated polynucleotide having at least 98 percent, and most preferably at least 99 percent, identity with any sequence of 100 to 1000 consecutive nucleotides of SEQ ID NOs: 3-5, or any complementary sequence of the foregoing.

In another aspect, the invention includes an isolated polynucleotide that forms a detectable duplex under stringent hybridization conditions with a polynucleotide having any sequence of 100 to 1000 consecutive nucleotides of SEQ ID NOs: 3-5, or any complementary sequence of the foregoing. In another aspect, the invention includes primer pairs for carrying out a PCR to amplify a segment of a polynucleotide of the invention. Each primer of a pair is an oligonucleotide having a length of between 15 and 30 nucleotides such that i) one primer of the pair forms a perfectly matched duplex with one strand of a polynucleotide of the invention and the other primer of the pair form a perfectly match duplex with the complementary strand of the same polynucleotide, and ii) the primers of a pair form such perfectly matched duplexes at sites on the polynucleotide that are separated by a distance of between 10 and 2500 nucleotides. Preferably, the annealing temperature of each primer of a pair with its respective complementary sequence is substantially the same.

In another aspect, the invention includes natural variants of the DJ11 nucleic acids having a frequency in a selected population of at least two percent. More preferably, such natural variant has a frequency in a selected population of at least five percent, and still more preferably, at least ten percent. Most preferably, such natural variant has a frequency in a selected population of at least twenty percent. The selected population may be any recognized population of study in the field of population genetics.

In another aspect, the invention provides a vector comprising DNA encoding an active (e.g. engineered) DJ11 polypeptide. The invention also includes host cells comprising such a vector. A process for producing a DJ11 polypeptide is also provided which comprises culturing the host cells under conditions suitable for expression of such polypeptide and its recovery from the cell culture materials. Another preferred method for producing a DJ11. polypeptide comprises the steps of: (a) providing a host cell capable of expressing DJ11; (b) culturing said host cell under conditions that allow expression of said DJ11; and (c) recovering said DJ11. Within one embodiment the expression vector further comprises a secretory signal sequence operably linked to the DNA segment, the cell secretes the protein into a culture medium, and the protein is recovered from the medium. An especially preferred method of producing DJ11 includes chemical synthesis using standard peptide synthesis techniques, as described in the section titled “Chemical Manufacture of DJ11 Compositions” and Example 1.

In another aspect, the invention includes polypeptides having a sequence which is at least 95 percent identical to SEQ ID NO: 1 or 2. Preferably, the invention includes polypeptides having at least 97 percent, and more preferably at least 98 percent, and still more preferably at least 99 percent, identity with any one of the sequences selected from the group consisting of SEQ ID NO: 1 and SEQ ID NO: 2. Most preferably, the invention includes polypeptides having a sequence at least 99 percent identical to SEQ ID NO: 1 or 2.

In another aspect, the invention includes an isolated peptide fragment of DJ11 having a sequence identical to a subsequence of SEQ ID NO: 1 or 2. Preferably, said fragment possesses at least one biological activity of the DJ11 polypeptide of SEQ ID NO: 1 or 2.

In another aspect, the invention includes an isolated peptide consisting of 6 to 40 amino acids whose sequence is identical to a subsequence of consecutive amino acids in the DJ11 polypeptide having the sequence of SEQ ID NOS 1 or 2. Such peptides are useful intermediates in the production of antigenic compositions used in the production of peptide antibodies specific for DJ11.

In another aspect, the invention includes isolated antibodies specific for any of the polypeptides or peptide fragments described above. Preferably, the antibodies of the invention are monoclonal antibodies. Preferably, an antibody of the invention is specific for a DJ11 polypeptide or antigenic fragment thereof and binds said DJ11 polypeptide with high affinity. Antibodies of the invention are advantageously attached to a label group to enable detection or purification. Anti-DJ11 antibodies may be used, for example, for detection, purification, or activity modulation of DJ11 polypeptides.

In another aspect, the invention provides a method of detecting a DJ11 biological activity comprising the steps of: i) providing a sample comprising a DJ11 polypeptide under conditions permissive to a DJ11 biological activity; and ii) measuring the level of DJ11 polypeptide activity in the sample. Preferably, the sample is a biological solution. Preferably, the DJ11 biological activity is serine protease inhibition.

In still a further aspect, the invention includes pharmaceutical compositions and formulations comprising a polypeptide having an amino acid sequence selected from the group consisting of SEQ ID NOS 1 and 2, or a fragment thereof, and a pharmaceutically acceptable carrier compound.

One aspect of the invention provides a method of decreasing serine protease activity comprising the step of contacting a DJ11 polypeptide or biologically active fragment thereof with a serine protease under conditions permissive to inhibition. Preferably, said contact takes place in a biological solution.

In another embodiment, the invention provides a method of identifying a modulator of at least one DJ11 biological activity comprising the steps of: i) contacting a test modulator of a DJ11 biological activity with the polypeptide comprising the amino acid sequence selected from the group consisting of SEQ ID NOs:1 and 2; ii) detecting the level of said DJ11 biological activity; and iii) comparing the level of said DJ11 biological activity to that of a control sample lacking said test modulator. Where the difference in the level of DJ11 protein biological activity is a decrease, the test modulator is an inhibitor of at least one DJ11 biological activity. Where the difference in the level of DJ11 biological activity is an increase, the test substance is an activator of at least one DJ11 biological activity. The invention further encompasses compounds thus identified, as well as compositions thereof.

### BRIEF DESCRIPTION OF THE FIGURE

FIG. 1 illustrates a multiple sequence alignment between human and mouse DJ11s (SEQ ID NO 15 and 16, respectively) and the human and mouse EPPIN proteins (SEQ ID NO 13 and 14, respectively). The two residues which have been modified in the engineered protein (at position 65 and 110) have been highlighted.

### BRIEF DESCRIPTION OF THE SEQUENCE LISTING

SEQ ID NO 1 is an amino acid sequence of an engineered human DJ11 protein.

SEQ ID NO 2 is an amino acid sequence of the mature form of an engineered human DJ11 protein.

SEQ ID NO 3 is the genomic DNA sequence of the DJ11 pseudogene.

SEQ ID NO 8 is a reconstructed cDNA sequence derived from the DJ11 pseudogene.

SEQ ID NO 11 is an engineered cDNA sequence encoding the human DJ11 protein of SEQ ID NO 1.

### DETAILED DESCRIPTION OF THE INVENTION

Definitions

The terms “polypeptide” or “peptide” or “peptide fragment” as used herein refer to compounds made up of a single unbranched chain of amino acid residues linked by peptide bonds. The number of amino acid residues in such compounds varies widely; however, preferably, peptides referred to herein usually have from six to forty amino acid residues. Peptide fragments preferably have from forty to eighty amino acid residues. Polypeptides referred to herein usually have from a few tens of amino acid residues, e.g. 20, to up to a few hundred amino acid residues, e.g. 200, or more.

The term “protein” as used herein may be used synonymously with the term “polypeptide” or may refer to, in addition, a complex of two or more polypeptides which may be linked by bonds other than peptide bonds, for example, such polypeptides making up the protein may be linked by disulfide bonds. The term “protein” may also comprehend a family of polypeptides having identical amino acid sequences but different post-translational modifications, such as phosphorylations, acylations, glycosylations, particularly as may be added when such proteins are expressed in eukaryotic hosts.

Amino acid residues are referred to herein by their standard single-letter or three-letter notations: A, alanine; C, cysteine; D, aspartic acid; E, glutamic acid; F, phenylalanine; G, glycine; H, histidine; I, Isoleucine; K, lysine; L, leucine; M, methionine; N, asparagine; P, proline; Q, glutamine; R, arginine; S, serine; T, threonine; V, valine; W, tryptophan; Y, tyrosine.

The term “DJ11” as used herein, encompasses polypeptides and proteins having an amino acid sequence of SEQ ID NOS 1 and 2, including natural variants and fragments thereof, whether prepared by recombinant or synthetic methods. Such polypeptide may be post-translationally modified as described herein. DJ11 may also contain other structural or chemical modifications such as disulfide linkages or amino acid side chain interactions such as hydrogen and amide bonds that result in complex secondary or tertiary structures. DJ11 also includes mutant polypeptides, such as deletion, addition, swap, or truncation mutants, fusion polypeptides comprising such polypeptides, and polypeptide fragments of at least three, but preferably 8, 10, 12, 15, or 21 contiguous amino acids of the sequence of SEQ ID NO:1 or 2. Further included are DJ11 proteolytic precursors and intermediates of the sequence selected from the group consisting of SEQ ID NOs:1-2. The invention embodies polypeptides encoded by the nucleic acid sequences of DJ11 genes or DJ11 mRNA species, preferably human DJ11 genes and mRNA species, including isolated DJ11 consisting of, consisting essentially of, or comprising the sequence of SEQ ID NO:1 or 2. Preferred DJ11 retain at least one biological activity of the DJ11 of SEQ ID NO:1 or 2. As used interchangeably herein, a “DJ11 protein” or “DJ11 polypeptide” may be any polypeptide encoded by a DJ11 nucleic acid, preferably a nucleic acid derived from the DJ11 pseudogene or transcript produced therefrom such as provided in SEQ ID NOs:3, 8 and 11. The term “DJ11 protein” or “DJ11 polypeptide” thus specifically includes the engineered biologically active DJ11 proteins. In preferred embodiments, a “DJ11” protein or “DJ11” polypeptide refers to a polypeptide having a DJ11 protease inhibitor domain as described herein, or a biologically active portion thereof.

As used interchangeably herein, a “DJ11 activity”, “biological activity of a DJ11 protein” or “functional activity of a DJ11 protein”, refers to an activity exerted by a DJ11 domain or DJ11 polypeptide or nucleic acid molecule, or a biologically active fragment or homologue thereof comprising a functional DJ11 domain as determined in vivo or in vitro, according to standard techniques. These include but are not limited to: (1) antigenicity, or the ability to bind an anti-DJ11 specific antibody; (2) immunogenicity, or the ability to generate an anti-DJ11 specific antibody; (3) forming intermolecular amino acid side chain interactions such as hydrogen, amide, or preferably disulfide links; (4) interaction with a DJ11 target molecule, preferably a serine protease; (5) inhibition of serine protease activity; and (6) reduction of transacylation catalysis by a serine protease. As used herein, transacylation refers to a reaction in which a leaving group is exchanged for a nucleophile. Alternatively, a DJ11 activity may be an indirect activity, such as an activity mediated by interaction of the DJ11 protein with a DJ11 target molecule that modulates a downstream cellular activity.

As used herein “effective amount” means an amount sufficient to ameliorate a symptom of a condition related to serine protease activity including any of the disorders described herein, but preferably the disorders described in the section titled “Therapeutic Applications of DJ11”. The effective amount for a particular patient may vary depending on such factors as the state of the condition being treated, the overall health of the patient, method of administration, and the severity of side-effects.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

“Percent identical” is used herein to refer to both nucleic acid sequences and amino acid sequences comparisons. To determine the percent identity (as used herein, “percent identity” is equivalent to “percent homology”) of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/(total # of positions×100)).

The comparison of sequences and determination of percent identity between two sequences can be accomplished using a mathematical algorithm. A preferred, non-limiting example of a mathematical algorithim utilized for the comparison of sequences is the algorithm of Karlin and Altschul ((1990) Proc. Natl. Acad. Sci. USA 87:2264-68), modified as in Karlin and Altschul ((1993) Proc. Natl. Acad. Sci. USA 90:5873-77), the disclosures of which are incorporated herein by reference in their entireties. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the sequences of the invention. BLAST protein searches can be performed with the XBLAST program, score=50, wordlength=3 to obtain amino acid sequences homologous to the polypeptide sequences of the invention. To obtain gapped alignments for comparison purposes, Gapped BLAST can be utilized as described in Altschul et al., ((1997) Nucleic Acids Research 25(17):3389-3402). When utilizing BLAST and Gapped BLAST programs, the default parameters of the respective programs (e.g., XBLAST and NBLAST) can be used. See http://www.ncbi.nlm.nih.gov, the disclosures of which are incorporated herein by reference in their entireties. Another preferred, non-limiting example of a mathematical algorithm utilized for the comparison of sequences is the algorithm of Myers and Miller, CABIOS (1989), the disclosures of which are incorporated herein by reference in their entireties. Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

The term “isolated” in reference to a polypeptide or polynucleotide of the invention means substantially separated from the components of its natural environment. Preferably, an isolated polypeptide or polynucleotide is a composition that consists of at least eighty percent of the polypeptide or polynucleotide identified by sequence on a weight basis as compared to components of its natural environment; more preferably, such composition consists of at least ninety-five percent of the polypeptide or polynucleotide identified by sequence on a weight basis as compared to components of its natural environment; and still more preferably, such composition consists of at least ninety-nine percent of the polypeptide or polynucleotide identified by sequence on a weight basis as compared to components of its natural environment. Most preferably, an isolated polypeptide or polynucleotide is a homogeneous composition that can be resolved as a single spot after conventional separation by two-dimensional gel electrophoresis based on molecular weight and isoelectric point. Protocols for such analysis by conventional two-dimensional gel electrophoresis are well known to one of ordinary skill in the art, e.g. Hames and Rickwood, Editors, Gel Electrophoresis of Proteins: A Practical Approach (IRL Press, Oxford, 1981); Scopes, Protein Purification (Springer-Verlag, New York, 1982); Rabilloud, Editor, Proteome Research: Two-Dimensional Gel Electrophoresis and Identification Methods (Springer-Verlag, Berlin, 2000).

The term “oligonucleotide” as used herein means linear oligomers of natural or modified monomers or linkages, including deoxyribonucleosides, ribonucleosides, anomeric forms thereof, peptide nucleic acids (PNAs), and the like, capable of specifically binding to a polynucleotide by way of a regular pattern of monomer-to-monomer interactions, such as Watson-Crick type of base pairing, base stacking, Hoogsteen or reverse Hoogsteen types of base pairing, or the like. Usually, monomers are linked by phosphodiester bonds, or analogs thereof, to form oligonucleotides ranging in size from a few monomeric units, e.g. 3-4, to several tens of monomeric units, e.g. 40-60. Whenever an oligonucleotide or polynucleotide is represented by a sequence of letters, such as “ATGCCTG,” or the lower case equivalent, it will be understood that the nucleotides are in 5′→3′ order from left to right and that “A” denotes deoxyadenosine, “C” denotes deoxycytidine, “G” denotes deoxyguanosine, “T” denotes thymidine, and “U” denotes uridine, unless otherwise noted or understood for their context. Usually oligonucleotides of the invention comprise the four natural nucleotides, and they are joined to one another by natural phosphodiester linkages; however, they may also comprise non-natural nucleotide analogs and may also contain non-natural inter-nucleosidic linkages, particularly when employed as antisense or diagnostic compositions. It is clear to those skilled in the art when oligonucleotides having natural or non-natural nucleotides may be employed in accordance with the invention, e.g. where processing by enzymes is called for, usually oligonucleotides consisting of natural nucleotides are required.

As used herein, “nucleoside” includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the only proviso that they are capable of specific hybridization. Such analogs include synthetic nucleosides designed to enhance binding properties, reduce complexity, increase specificity, and the like.

As used herein, “detectable duplex” in reference to a hybridization assay means that for any type of signal generating means used the signal-to-noise ratio is at least two. Preferably, such signal-to-noise ratio is at least three, and more preferably, such signal-to-noise ratio is at least five.

For purposes of this invention, “pseudogene” is meant to include transcriptionally and translationally inactive sequences, homologous to a corresponding functional gene from which these pseudogenes may have arisen, which can be distinguished from the cognate functional gene on the basis of mutational differences in the sequences, at least some of such mutational sequences differences would result in premature termination of translation due to the presence of codon sequences that signal translational termination.

Kunitz Domain Inhibitors

Protein inhibitors of serine proteinases can be grouped into several families, including the Kunitz, serpin, Kazal, and mucous protein inhibitor families, based on conserved structural features. Members of each family exhibit greatly varied binding specificities, and members of different families can have similar substrate profiles. The binding specificities of proteinase inhibitors are determined by the residue at the P1 position as well as other residues located at the interface between the inhibitor and the bound target proteinase. The P1 residue in Kunitz domain proteins is immediately C-terminal to the conserved second cysteine (position 15 using aprotinin numbering; amino acid 87 in SEQ ID NO:1).

Members of the Kunitz domain protein family function as inhibitors of serine proteases. Each inhibitor has a unique substrate profile. However, inhibitors with a basic residue (i.e., arginine or lysine) immediately following the second cysteine residue generally inhibit proteases that cleave proteins at basic residues. In addition, mutation of the lysine residue at this position in aprotinin to a valine resulted in a dramatic increase in the protein's potency towards neutrophil elastase, a protease that typically cleaves proteins at residues with small neutral aliphatic side chains (Roberts, L. et al., 1992, PNAS 89:2429-33).

The serine protease inhibitory activities of the Kunitz domain proteins has led to their evaluation as potential therapeutics in a number of disease indications. For example, aprotinin is a potent inhibitor of proteases involved in the blood clotting cascade and is used clinically to reduce bleeding during open heart surgery (Davis, R. & Whittington, R., 1995, Drugs 49:954-983). Human placental bikunin is a potent inhibitor of plasmin, which has been implicated in facilitating metastasis and tumor growth (Meissauer, A. et al., 1991, Exp. Cell Res. 192:453-9). Other disease indications in which serine proteases are believed to play a significant pathological role and I n which the Kunitz domain proteins may therefore be effective therapeutics include traumatic brain injury and stroke (Greenwood, J., 1991, Neuroradiology 33:95-100; Coleman, 1984, J. Clin. Invest. 73:1249), cystic fibrosis (Vallet et al., Nature 389:607; Chraibi et al., J. Gen. Physiol., 111:127), emphysema (Huang, Y. et al., 1998, J. Leukoc. Biol. 64: 322-30), arthritis and anemia (Dela Cadena, R. et al., 1995, FASEB J. 9:446-452) and non-insulin dependent diabetes (Laurenti et al., 1996, Diabetic Medicine 13:642-645).

Most Kunitz family serine proteinase inhibitors exhibit significantly tighter binding to trypsin and chymotrypsin than to the three neutral proteinases secreted by neutrophils. These two proteases have relatively strict P1 specificities (trypsin=arginine, lysine; chymotrypsin=tyrosine, phenylalanine, tryptophan) but few restrictions at other P and P′ positions. For example, aprotinin is a potent inhibitor of trypsin (Ki=0.02 nM) and chymotrypsin (Ki=1.3 nM) but does not inhibit leukocyte elastase (Delaria et al., 1997, J. Biol. Chem. 272:12209-12). Similarly, placental bikunin inhibits trypsin (Ki=0.01 nM) and chymotrypsin (Ki=0.48 nM) but not leukocyte elastase (Delaria et al, supra). Tissue factor pathway inhibitor (TFPI), another member of the Kunitz family, inhibits trypsin (0.1 nM) and chymotrypsin (Ki=0.75 nM) but is a weak inhibitor of leukocyte elastase (Ki=400 nM) and cathepsin G (Ki=100-200 nM) (Petersen et al., Eur J Biochem, 1996;235(1-2):310-6; Petersen et al., Thromb Haemost May 4, 1992;67(5):537-41). In addition, these Kunitz family members exhibit potent inhibitory activity towards serine proteinases having trypsin-like substrate specificity involved in both coagulation and fibrinolysis. Elastase and cathepsin G have been reported to proteolytically cleave and inactivate TFPI (Petersen et al., 1992).

Human inter-alpha-trypsin inhibitor (I alpha I), a plasma Kunitz family proteinase inhibitor, is a potent inhibitor of trypsin (Ki=0.078 nM) and chymotrypsin (1.1 nM) but exhibits somewhat lesser activity against cathepsin G (Ki=18 nM) and leukocyte elastase (Ki=61 nM) (Swaim and Pizzo. Biochem J, 1988;254(1):171-8). Similarly, a Kunitz-type inhibitor purified from Japanese horseshoe crab (Tachypleus tridentatus) hemocytes potently inhibited trypsin (Ki=0.46 nM) and chymotrypsin (Ki=5.5 nM), but was somewhat less active towards leukocyte elastase (Ki=72 nM) (Nakamura et al., J Biochem (Tokyo), 1987; 101(5):1297-306).

Soybean trypsin inhibitor (STI) is a potent Kunitz family inhibitor of trypsin but a significantly weaker inhibitor of chymotrypsin (Ki(1)=1000 nM; Ki(2)=300 nM) (Bosterling and Quast, Biochim Biophys Acta, 1981;657(1):58-72). STI has been reported to exhibit similar inhibitory activity towards chymotrypsin and leukocyte elastase (Fagny et al., Regul Pept, 1992;42(1-2):27-37). On the other hand, a serine protease inhibitor from larvae of parasitic nematode Anisakis simplex that has 96% amino acid identity to soybean trypsin inhibitor was reported to inhibit trypsin and elastase but not chymotrypsin (Morris and Sakanari, J Biol Chem, 1994;269(44):27650-6). In addition, a Kunitz-type inhibitor purified from potato tubers (Solanum tuberosum L.) was reported to be an effective inhibitor of trypsin, leukocyte elastase, and chymotrypsin (Valueva et al., Biochemistry (Mosc) 1997 62(12):1367-74).

Methods of Designing an Engineered Kunitz-Type Protease Inhibitor

The method of the invention provides for production of engineered proteases through a process of designing a protease inhibitor peptide or nucleic acid based on a pseudogene nucleic acid sequence. In one aspect, the invention encompasses a method comprising the steps of: comparing a nucleic acid sequence derived from a pseudogene to a nucleic acid sequence of one or more known protease inhibitors, protease inhibitor domains, or related consensus or sequence signatures, and providing a modified nucleic acid sequence encoding an active protease inhibitor, or an amino acid sequence of an active protease inhibitor. Preferably the protease inhibitor will be a Kunitz-type protease inhibitor, or more preferably a DJ11 protease inhibitor. Preparing an engineered protease inhibitor according to the invention can be carried out using the process of site specific and/or random site mutagenesis of the pseudogene nucleic acid sequence. Alternatively, the method of the invention provides a means of producing an engineered protease through the process of traditional mutagenesis. The invention further provides for selection and screening of a suitably modified engineered protease that is capable of binding or interacting with a protease, preferably a serine protease, and preferably inhibiting the catalysis of a transacylation reaction carried out by said serine protease, most preferably a preselected transacylation reaction. The invention also provides a process for utilizing the engineered protease inhibitor to prevent or decrease the transacylation of a preselected protease substrate, to modulate the level of said substrate.

Engineered protease inhibitors are rendered suitable for effective inhibition of a protease by modifying a defective protease inhibitor gene such that the encoded protein has at least the minimum domain necessary for protease inhibition.

Engineered protease inhibitors can further be rendered suitable for effective inhibition of a protease through modification of the defective protease inhibitor gene at the “active site.” Modifications of the engineered protease inhibitor can be site specific mutations designed to alter the “active site” of the protease inhibitor so that it can act upon different substrates (e.g., preselected proteases) than known protease inhibitors. Modifications can include substitution, deletion, or insertion of one or more amino acids. These modifications can also be generated by random mutagenesis.

Kunitz-type protease inhibitor active sites are known to those of skill in the art. In addition, amino acids equivalent to those in known binding sites can be identified using standard methods (e.g., reference to the primary and/or tertiary structure of a polypeptide in that class of protease inhibitors). For example, a reference polypeptide for the DJ11 protease inhibitor domain is Eppin protein (Epididymal protease inhibitor, available from SwissProt at http://wwv.expasy.ch, under the accession number 095925, and also as SEQ ID NO:13 herein) or bovine pancreatic trypsin inhibitor (BPTI). The primary amino acid sequence and the crystal structure of BPTI are known and serve as reference points to identify equivalent amino acids in DJ11, as described herein.

Generally, the Kunitz-type protease inhibitor domain can be modified accordingly, using examples of mammalian Kunitz-type inhibitors such as BPTI, Alzheimer's amyloid beta-protein precursor, and inter-alpha-trypsin inhibitor (Creighton, T. E. and I. G. Charles, Cold Spring Harbor Symp. Quant. Biol. 52: 511-519, 1987; Salvesen, G. and Pizzo, S., in Hemostasis and Thrombosis: Basic Principles and Clinical Practice (Colman, R. W., Hirsh, J., Marder, V., & Salzman, E. W., eds.) pp. 241-258, J. B. Lippincott Co., Philadelphia, 1994). Kunitz-type protease inhibitors have also been prepared from the alpha-3 chain of human type VI collagen (see WO 93/14119). They have also been identified in many snake venoms. Kunitz inhibitors of TF-FVIIa have also been prepared from BPTI using phage display technology (De Maeyer et al., Thrombosis and Haemostasis Abstracts, XIVth Congress of the International Society on Thrombosis and Haemostasis, p 888 Ab. No. 1245, 1993), reporting a mutant BPTI (Thr11Asp, Lys15Arg, Arg17Leu, Ile18His, Ile19Leu, Val34Tyr, Arg39Leu and Lys46Glu) having a Ki for TF-FVIIa of 0.5 nM.

The crystal structures of Kunitz domains reveal key residues likely to make contact with the serine protease domain of serine proteases (Hynes, T. R. et al., Biochemistry 29: 10018-10022,1990; Bode, W. and Huber, R., Eur. J. Biochem. 204: 433-451,1992; Kossiakoff, A. A. et al., Biochem Soc Trans 21: 614-618, 1993). Although the amino acid at the P1 position generally dominates the affinity of inhibitors for the serine protease active site (Scott, C. F. et al., Blood 69: 1431-1436, 1987; Laskowski, M., Jr. and Kato, I., Annu. Rev. Biochem. 49: 593-626, 1980; Beckmann, J. et al., Eur. J. Biochem. 176: 675-682,1988; Sinha, S. et al, J. Biol. Chem. 266: 21011-21013, 1991), residues outside this region are also known to play a role in binding affinity and specificity towards serine proteases (Kossiakoff, A. A. et al., Biochem. Soc. Trans. 21: 614-618, 1993; Roberts, B. L. et al, Proc Natl Acad Sci USA 89: 2429-2433, 1992). Some of the contact residues in the binding loop (positions 11, 15, 17, and 19) are relatively variable among Kunitz domains (Creighton, T. E. and I. G. Charles, Cold Spring Harbor Symp. Quant. Biol. 52: 511-519, 1987). Position 13 (amino acid 85 in SEQ ID NO:1) is normally a Pro; however, other residues are sometimes found here. Position 12 (amino acid 84 in SEQ ID NO:1) is almost always a Gly. In addition to recruiting any side chain interactions, substitution of other residues for Pro and vice versa might also lead to conformational changes in the main chain which could affect binding. The cysteine residues at positions 14 and 38 (positions 86 and 110 in SEQ ID NO:1) that form a disulfide bond are always found in Kunitz domains; however other residues such as Ala, Gly, Ser, or Thr may substitute for the cysteines (Marks, C. B. et al., Science, 235: 1370-1373, 1987). Other residues are important as well; BPTI for example has a methionine at position 52, although other Kunitz domains such as human DJ11 have a variety of residues at this position. Methionine at this position can be replaced by different residues which may be beneficial with respect to producing the protein. Substitutions at position 52 are not expected to have major effects on inhibitory activity since it is so far away from the primary binding loop of the Kunitz domain.

Thus, once a pseudogene-derived sequence has been modified to encode a polypeptide comprising at least one effective protease inhibitor domain, residues in the active site of the protease inhibitor with an unknown tertiary structure are then compared to active site residues of a homologous protease inhibitor with a known tertiary structure. This method can thus be used to identify amino acids in the protease inhibitor of interest that are equivalent to amino acids in the active site of the homologous protease inhibitor (Olesen et al. Protein Engineering, 6:409 (1993)).

Upon identification of residues required for protease inhibitor activity and design of an engineered protease inhibitor, a protease inihibitor polypeptide can be prepared using site specific mutagenesis based on the pseudogene cDNA sequence. By way of illustration, with expression vectors encoding DJ11 in hand, site specific mutagenesis (Kunkel et al., Methods Enzymol. 204:125-139, 1991; Carter, P., et al., Nucl. Acids. Res. 13:4331, 1986; Zoller, M. J. et al., Nucl. Acids Res. 10:6487, 1982), cassette mutagenesis (Wells, J. A., et al., Gene 34:315, 1985), restriction selection mutagenesis (Wells, J. A., et al., Philos. Trans, R. Soc. London SerA 317, 415, 1986) or other known techniques maybe performed on the DJ11 DNA. The mutant DNA encoding a functional protease inhibitor may be used, for example, in place of the parent DNA, by insertion into the aforementioned expression vectors. Growth of host bacteria containing the expression vectors with the mutant DNA allows the production of mutant DJ11 (i.e., analogs or homologs of DJ11), which can be isolated as described herein.

The mutant or engineered protease inhibitor can then be tested using known protease inhibition assays as further described herein. Preferably an engineered inhibitor will have enhanced protease inhibition capability, including enhanced specificity or efficiency in inhibiting a protease over a known protease inhibitor.

It will be appreciated that the nucleic acid sequence of any suitable protease inhibitor pseudogene can be used as the starting point for design of a biologically active protease inhibitor. In a preferred embodiment, the invention provides a method for designing or preparing a biologically active protease inhibitor based on a Kunitz-type protease inhibitor pseudogene-derived nucleic acid sequence, more preferably an EPPIN or DJ11 protease inhibitor pseudogene-derived nucleic acid sequence, or preferably the nucleic acid sequence of SEQ ID NO: 3 or 8.

Native and Engineered DJ11 Nucleic Acids

The human DJ11 gene is localized at chromosome 20q12-13.2. The nucleic acid sequence of the human DJ11 pseudogene is shown in SEQ ID NO 3, while a reconstructed cDNA sequence from the DJ11 pseudogene is shown in SEQ ID NO 8. The sequence of a modified DJ11 cDNA encoding a preferred active DJ11 protein is shown in SEQ ID NO 11. One aspect of the invention thus pertains to purified or isolated nucleic acid molecules that encode DJ11 proteins or biologically active portions thereof as further described herein, as well as nucleic acid fragments thereof. Said nucleic acids may be used for example in therapeutic, diagnostic, research and industrial methods as further described herein.

One object of the invention is a purified, isolated, or recombinant nucleic acid comprising, consisting essentially of, or consisting of the nucleotide sequence of SEQ ID NOS 3, 8 or 11, complementary sequences thereto, and fragments thereof. The invention also pertains to a purified or isolated nucleic acid comprising a polynucleotide having at least 95% nucleotide identity with a polynucleotide of SEQ ID NOS 3, 8 or 11, advantageously 99% nucleotide identity, preferably 99.5% nucleotide identity and most preferably 99.8% nucleotide identity with a polynucleotide of SEQ ID NOS 3, 8 or 11, or a sequence complementary thereto or a fragment thereof Another object of the invention relates to purified, isolated or recombinant nucleic acids comprising a polynucleotide that hybridizes, under the stringent hybridization conditions defined herein, with a polynucleotide of SEQ ID NOS 3, 8 or 11, or a sequence complementary thereto or a variant thereof or a fragment thereof. In further embodiments, nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, 1000 or 1300 nucleotides of SEQ ID NOS 3, 8 or 11, or the complements thereof. In yet further embodiments, said contiguous span comprises at least one nucleotide of nucleotide positions 1 to 75 of SEQ ID NO 11.

The DJ11 pseudogene derived EDNA of SEQ ID NO: 8 may be used to prepare a nucleic acid encoding a biologically active DJ11 protein as further described herein. In preferred embodiments, the invention encompasses a DJ11 nucleic acid sequence comprising a contiguous span of at least 12, 15, 18, 20, 25, 30, 35, 40, 50, 60, 70, 80, 90, 100, 150, 200, 250, 275 or 290 nucleotides of SEQ ID NO: 8, or the complements thereof, wherein said contiguous span comprises at least one nucleotide selected from nucleotide positions 1 to 294 and 296 to 394 of SEQ ID NO: 8.

In a further aspect, the invention encompasses identifying the 4 exons of the DJ11 pseudogene of SEQ ID NO: 3. In preferred aspects, the invention encompasses a contiguous span of at least 12, 15, 18, 20, 25, 30, 35, 40, 50, 60, 70, 80, 90, 100, 150, 200, 500, 1000, 2000, 5000, 8000, or 10000 nucleotides of SEQ ID NO: 3, or the complements thereof. In particularly preferred embodiments, the invention encompasses a contiguous span of at least 12, 15, 18, 20, 25, 30, 35, 40, 50, 60, 70, 80, 90 or 100 nucleotides of SEQ ID NO: 3, or the complements thereof, wherein said contiguous span comprises at least one nucleotide selected from nucleotide positions 2043 to 2132, 3358 to 3486, 6120 to 6191, and 6193 to 6291 of SEQ ID NO: 3.

Also encompassed is a purified, isolated, or recombinant nucleic acid polynucleotide encoding a DJ11 polypeptide of the invention, as further described herein.

In another preferred aspect, the invention pertains to purified or isolated nucleic acid molecules that encode a portion or variant of a DJ11 protein, wherein the portion or variant displays a DJ11 activity of the invention. In other embodiments, the invention relates to a polynucleotide encoding a DJ11 portion consisting of at least 8-20, 20-50, 50-70, 60-100, 100, 120 or 130 amino acids of SEQ ID NOS 1 or 2, or a variant thereof, wherein said DJ11 portion displays a DJ11 activity described herein.

The sequence of SEQ ID NO: 11 is a cDNA encoding a selected engineered human DJ11 protease. Also encompassed by the DJ11 gene and DJ11 nucleic acids of the invention are nucleic acid molecules which are complementary to a DJ11 gene, DJ11 cDNA, or DJ11 nucleic acids described herein. Preferably, a complementary nucleic acid is sufficiently complementary to the nucleotide sequence shown in SEQ ID NOS 3, 8 or 11, such that it can hybridize to the nucleotide sequence shown in SEQ ID NOS 3, 8 or 11, thereby forming a stable duplex. Preferably, said complementarty nucleic acid is complementary to the polynucleotide of SEQ ID NO: 11 and hybridizes to the polynucleotide of SEQ ID NO: 11 under high stringency conditions.

Another object of the invention is a purified, isolated, or recombinant nucleic acid encoding a DJ11 polypeptide comprising, consisting essentially of, or consisting of the amino acid sequence of SEQ ID NOS 1 or 2, or fragments thereof, wherein the isolated nucleic acid molecule encodes one or more motifs selected from the group consisting of a Kunitz-type protease inhibitor domain, a WAP domain (InterPro ref. IPR002221), and a signal sequence. For example, the purified, isolated or recombinant nucleic acid may comprise a genomic DNA or fragment thereof which encodes the polypeptide of SEQ ID NOS 1 or 2 or a fragment thereof or a cDNA consisting of, consisting essentially of, or comprising the sequence of SEQ ID NOS 8 or 11 or fragments thereof, wherein the isolated nucleic acid molecule encodes one or more motifs selected from the group consisting of a Kunitz-type protease inhibitor domain, a WAP domain and a signal sequence. Any combination of said motifs may also be specified.

The invention further encompasses nucleic acid molecules that differ from the DJ11 or DJ11 nucleotide sequences of the invention due to degeneracy of the genetic code and encode the same DJ11 proteins and fragment of the invention.

In addition to the DJ11 nucleotide sequences described above, it will be appreciated by those skilled in the art that DNA sequence polymorphisms that lead to changes in the amino acid sequences of the DJ11 proteins may exist within a population (e.g., the human population). Such genetic polymorphism may exist among individuals within a population due to natural allelic variation. Such natural allelic variations can typically result in 1-5% variance in the nucleotide sequence of the DJ11 gene or nucleic acid sequence encoding DJ11 polypeptide.

Nucleic acid molecules corresponding to natural allelic variants and homologues of the DJ11 nucleic acids of the invention can be isolated based on their homology to the DJ11 nucleic acids disclosed herein using the cDNAs disclosed herein, or a portion thereof, as a hybridization probe according to standard hybridization techniques under stringent hybridization conditions.

The stringency of a hybridization reaction can be controlled using well known techniques, e.g. by varying temperature, salt concentration, concentration of organic solvents, such as formamide (Britten et al, Meth. in Enzymol., 29: 363-418 1974; Breslauer et al, Proc. Natl. Acad. Sci., 83: 3746-3750 1986; Wetmur, Critical Reviews in Biochemistry and Molecular Biology, 26: 227-259 1991; Keller et al, DNA Probes, Second Edition (Stockton Press, New York, 1993)). The following exemplary hybridization stringency conditions are preferably used to identify polynucleotides within the scope of the invention: (1) probe DNA (1-15 ng/μL) hybridizes to target DNA at about 65° C. in about 5×SSPE and is washed under conditions of about 65° C. in about 0.1×SSPE (see Sambrook, et al., MOLECULAR CLONING: A LABORATORY MANUAL, 2d Edition (Cold Spring Harbor Laboratory, New York,1989), and Ausubel, F. M., et al., Eds., CURRENT PROTOCOLS IN MOLECULAR BIOLOGY, (John Wiley & Sons, Inc., New York, 1990)); and more preferably, (2) for 1-15 ng/μL of isolated nucleic acid probe hybridizing to a target polynucleotide bound to a nitrocellulose filter, hybridization in 25 mM KPO₄ (pH 7.4), 5×SSC, 5×Denhart's solution, 50 mg/mL denatured sonicated salmon sperm DNA, 50% formamide, 10% Dextran sulfate at 42° C., with washes at 65° C. in 2×SSC and 0.1% SDS. Preferably, the wash times in the above assays are 15 minutes, and more preferably, 30 minutes, and most preferably, 1 hour.

Uses of DJ11 Polynucleotides

Polynucleotide sequences (or the complements thereof) which encode DJ11 polypeptides have various applications, including uses as hybridization probes, in chromosome and gene mapping, and in the generation of antisense RNA and DNA. In addition, DJ11-encoding nucleic acids are useful as targets for pharmaceutical intervention, e.g. for the development of DNA vaccines, and for the preparation of DJ11 polypeptides by recombinant techniques, as described herein. The polynucleotides described herein, including sequence variants thereof, can be used in diagnostic assays. Accordingly, diagnostic methods based on detecting the presence of such polynucleotides in body fluids or tissue samples are a feature of the present invention. Examples of nucleic acid based diagnostic assays in accordance with the present invention include, but are not limited to, hybridization assays, e.g., in situ hybridization, and PCR-based assays. Polynucleotides, including extended length polynucleotides, sequence variants and fragments thereof, as described herein, may be used to generate hybridization probes or PCR pruners for use in such assays. Such probes and primers will be capable of detecting polynucleotide sequences, including genomic sequences that are similar, or complementary to, the DJ11 polynucleotides described herein. Detection of the DJ11 polynucleotide of SEQ ID NO: 11 may be used for example, to monitor expression of DJ11 in an exogenous cell host or of therapeutically-applied DJ11 polynucleotides in an individual.

The invention includes primer pairs for carrying out a PCR to amplify a segment of a polynucleotide of the invention. Each primer of a pair is an oligonucleotide having a length of between 15 and 30 nucleotides such that i) one primer of the pair forms a perfectly matched duplex with one strand of a polynucleotide of the invention and the other primer of the pair form a perfectly matched duplex with the complementary strand of the same polynucleotide, and ii) the primers of a pair form such perfectly matched duplexes at sites on the polynucleotide that are separated by a distance of between 10 and 2500 nucleotides. Preferably, the annealing temperature of each primer of a pair to its respective complementary sequence is substantially the same.

Hybridization probes derived from polynucleotides of the invention can be used, for example, in performing in situ hybridization on tissue samples, such as fixed or frozen tissue sections prepared on microscopic slides or suspended cells. Briefly, a labeled DNA or RNA probe is allowed to bind its DNA or RNA target sample in the tissue section on a prepared microscopic slide, under controlled conditions. Generally, dsDNA probes consisting of the DNA of interest cloned into a plasmid or bacteriophage DNA vector are used for this purpose, although ssDNA or ssRNA probes may also be used. Probes are generally oligonucleotides between about 15 and 40 nucleotides in length. Alternatively, the probes can be polynucleotide probes generated by PCR random priming primer extension or in vitro transcription of RNA from plasmids (riboprobes). These latter probes are typically several hundred base pairs in length. The probes can be labeled by any of a number of methods, including fluorescent tags, enzymes or radioactive moieties, according to methods well known in the art. The particular detection method will correspond to the type of label utilized on the probe (e.g., autoradiography, X-ray detection, fluorescent or visual microscopic analysis, as appropriate). The reaction can be further amplified in situ using immunocytochemical techniques directed against the label of the detector molecule used, such as antibodies directed to a fluorescein moiety present on a fluorescently labeled probe, or against avidin, or marker enzymes (peroxidase, alkaline phosphatase). Specific labeling and in situ detection methods can be found, for example, in Howard, G. C., Ed., Methods in Nonradioactive Detection, Appleton & Lange, Norwalk, Conn., (1993), herein incorporated by reference.

PCR in situ hybridization of tissue sections and/or cell samples provides a highly sensitive detection method for rare cell types in fixed cell or tissue samples. The PCR in situ hybridization detection method is carried out in accordance with methods that are known in the art, e.g., Nuovo, G. J., PCR IN SITU HYBRIDIZATION: PROTOCOLS AND APPLICATIONS, Raven Press, N.Y., 1992; U.S. Pat. No. 5,538,871, both of which are incorporated herein by reference.

Nucleotide sequences encoding a DJ11 polypeptide can also be used to construct hybridization probes for mapping the gene which encodes that DJ11 and for the genetic analysis of individuals. Individuals carrying variations of, or mutations in the gene encoding a DJ11 of the present invention may be detected at the DNA level by a variety of techniques. Nucleic acids used for diagnosis may be obtained from a patient's cells, including, for example, tissue biopsy and autopsy material. Genomic DNA may be used directly for detection or may be amplified enzymatically by using PCR prior to analysis (Saiki, et al. Nature 324:163-166 (1986)). RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype. Point mutations can be identified by hybridizing amplified DNA to radiolabeled RNA of the invention or alternatively, radiolabeled antisense DNA sequences of the invention. Sequence changes at specific locations may also be revealed by nuclease protection assays, such RNase and S1 protection or the chemical cleavage method e.g. Cotton, et al., Proc. Natl. Acad. Sci. USA 85:4397-4401 (1985), or by differences in melting temperatures. “Molecular beacons” (Kostrikis L. G. et al., Science 279:1228-1229 (1998)), hairpin-shaped, single-stranded synthetic oligonucleotides containing probe sequences which are complementary to the nucleic acid of the present invention, may also be used to detect point mutations or other sequence changes as well as monitor expression levels of DJ11.

Secretion Vectors and Recombinant Manufacture of DJ11 Compositions

The polynucleotide sequences described herein can be used in recombinant DNA molecules that direct the expression of the corresponding polypeptides in appropriate host cells. Because of the degeneracy in the genetic code, other DNA sequences may encode the equivalent amino acid sequence, and may be used to clone and express the DJ11 polypeptides. Codons preferred by a particular host cell may be selected and substituted into the naturally occurring nucleotide sequences, to increase the rate and/or efficiency of expression. The nucleic acid (e.g., cDNA or genomic DNA) encoding the desired DJ11 polypeptide may be inserted into a replicable vector for cloning (amplification of the DNA), or for expression.

The DJ11 cDNAs of SEQ ID NOS 8 or 11, or the DJ11 genomic DNAs of SEQ ID NO 3 may also be used to construct secretion vectors capable of directing the secretion of the proteins encoded by genes inserted in the vectors. Such secretion vectors may facilitate the purification or enrichment of the proteins encoded by genes inserted therein by reducing the number of background proteins from which the desired protein must be purified or enriched. Exemplary secretion vectors are described below.

The secretion vectors of the present invention include a promoter capable of directing gene expression in the host cell, tissue, or organism of interest. Such promoters include the Rous Sarcoma Virus promoter, the SV40 promoter, the human cytomegalovirus promoter, and other promoters familiar to those skilled in the art.

A polynucleotide of the invention encoding a signal sequence is operably linked to the promoter such that the mRNA transcribed from the promoter will direct the translation of the signal peptide. The host cell, tissue, or organism may be any cell, tissue, or organism which recognizes the signal peptide encoded by the signal sequence in the cDNA or genomic DNA of the invention. Suitable hosts include mammalian cells, tissues or organisms, avian cells, tissues, or organisms, insect cells, tissues or organisms, or yeast. Preferably a signal sequence comprises a contiguous span of at least 6, 8, 10, 12, 15, 20, 22 or 25 amino acid residues, said contiguous span comprising at least one amino acid residue of positions 1 to 25 of SEQ ID NO 1. A preferred polynucleotide encoding a signal sequence comprises a contiguous span of at least 15, 20, 30, 40, 50, 60, 70 or 75 nucleotides, said contiguous span comprising at least one nucleotide of nucleotide positions 1 to 75 of SEQ ID NO 11.

In addition, the secretion vector contains cloning sites for inserting genes encoding the proteins which are to be secreted. The cloning sites facilitate the cloning of the insert gene in frame with the signal sequence such that a fusion protein in which the signal peptide is fused to the protein encoded by the inserted gene is expressed from the mRNA transcribed from the promoter. The signal peptide directs the extracellular secretion of the fusion protein. The inserted gene may be a gene encoding a DJ11 polypeptide, or may be a gene heterologous to the DJ11 signal sequence.

The secretion vector may be DNA or RNA and may integrate into the chromosome of the host, be stably maintained as an extrachromosomal replicon in the host, be an artificial chromosome, or be transiently present in the host. Preferably, the secretion vector is maintained in multiple copies in each host cell. As used herein, multiple copies means at least 2, 5, 10, 20, 25, 50 or more than 50 copies per cell. In some embodiments, the multiple copies are maintained extrachromosomally. In other embodiments, the multiple copies result from amplification of a chromosomal sequence.

Many nucleic acid backbones suitable for use as secretion vectors are known to those skilled in the art, including retroviral vectors, SV40 vectors, Bovine Papilloma Virus vectors, yeast integrating plasmids, yeast episomal plasmids, yeast artificial chromosomes, human artificial chromosomes, P element vectors, baculovirus vectors, or bacterial plasmids capable of being transiently introduced into the host.

The secretion vector may also contain a polyA signal such that the polyA signal is located downstream of the gene inserted into the secretion vector.

After the gene encoding the protein for which secretion is desired is inserted into the secretion vector, the secretion vector is introduced into the host cell, tissue, or organism using calcium phosphate precipitation, DEAE-Dextran, electroporation, liposome-mediated transfection, viral particles or as naked DNA. The protein encoded by the inserted gene is then purified or enriched from the supernatant using conventional techniques such as ammonium sulfate precipitation, immunoprecipitation, immunochromatography, size exclusion chromatography, ion exchange chromatography, and hplc. Alternatively, the secreted protein may be in a sufficiently enriched or pure state in the supernatant or growth media of the host to permit it to be used for its intended purpose without further enrichment.

The signal sequences may also be inserted into vectors designed for gene therapy. In such vectors, the signal sequence is operably linked to a promoter such that mRNA transcribed from the promoter encodes the signal peptide. A cloning site is located downstream of the signal sequence such that a gene encoding a protein whose secretion is desired may readily be inserted into the vector and fused to the signal sequence. The vector is introduced into an appropriate host cell. The protein expressed from the promoter is secreted extracellularly, thereby producing a therapeutic effect.

The polypeptide can be expressed recombinantly in any of a number of expression systems according to methods known in the art (Ausubel, et al., editors, Current Protocols in Molecular Biology, John Wiley & Sons, New York, 1990). Appropriate host cells include yeast, bacteria, archebacteria, fungi, and insect and animal cells, including mammalian cells, for example primary cells, including stem cells, including, but not limited to bone marrow stem cells. More specifically, these include, but are not limited to, microorganisms such as bacteria transformed with recombinant bacteriophage, plasmid or cosmid DNA expression vectors, and yeast transformed with yeast expression vectors. Also included, are insect cells infected with a recombinant insect virus (such as baculovirus), and mammalian expression systems. The nucleic acid sequence to be expressed may be inserted into the vector by a variety of procedures. In general, DNA is inserted into an appropriate restriction endonuclease site using techniques known in the art. Vector components generally include, but are not limited to, one or more of a signal sequence, an origin of replication, one or more marker genes, an enhancer element, a promoter, and a transcription termination sequence. Constriction of suitable vectors containing one or more of these components employs standard ligation techniques which are known to the skilled artisan.

The DJ11 proteins of the present invention are produced by culturing a host cell transformed with an expression vector containing a nucleic acid encoding a DJ11, under the appropriate conditions to induce expression of the protein. The conditions appropriate for DJ11 expression will vary with the choice of the expression vector and the host cell, as ascertained by one skilled in the art. For example, the use of constitutive promoters in the expression vector may require routine optimization of host cell growth and proliferation, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

A host cell strain may be chosen for its ability to modulate the expression of the inserted sequences or to process the expressed protein in the desired fashion. Such modifications of the protein include, but are not limited to, glycosyl, acetyl, phosphate, amide, lipid, carboxyl, acyl, or carbohydrate groups. Post-translational processing, which cleaves a “prepro” form of the protein, may also be important for correct insertion, folding and/or function. By way of example, host cells such as CHO, HeLa, BHK, MDCK, 293, W138, etc. have specific cellular machinery and characteristic mechanisms for such post-translational activities and may be chosen to ensure the correct modification and processing of the introduced, foreign protein. Of particular interest are Drosophila melangaster cells, Saccharomyces cerevisiae and other yeasts, E. coli, Bacillus subtilis, SF9 cells, Cl29 cells, 293 cells, Neurospora, BHK, CHO, COS, and HeLa cells, fibroblasts, Schwanoma cell lines, immortalized mammalian myeloid and lymphoid cell lines, Jukat cells, human cells and other primary cells.

The nucleic acid encoding a DJ11 must be “operably linked” by placing it into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” DNA sequences are contiguous, and, in the case of a secretory leader or other polypeptide sequence, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Promoter sequences encode either constitutive or inducible promoters. The promoters may be either naturally occurring promoters or hybrid promoters. Hybrid promoters, which combine elements of more than one promoter, are also known in the art, and are useful in the present invention. The expression vector may comprise additional elements, for example, the expression vector may have two replication systems, thus allowing it to be maintained in two organisms, for example in mammalian or insect cells for expression and in a procaryotic host for cloning and amplification. Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2: plasmid origin is suitable for yeast, and various viral origins (SV40, polyoma, adenovirus, VSV or BPV) are useful for cloning vectors in mammalian cells. Further, for integrating expression vectors, the expression vector contains at least one sequence homologous to the host cell genome, and preferably, two homologous sequences which flank the expression construct. The integrating vector may be directed to a specific locus in the host cell by selecting the appropriate homologous sequence for inclusion in the vector. Constructs for integrating vectors are well known in the art. In an additional embodiment, a heterologous expression control element may be operably linked with the endogenous gene in the host cell by homologous recombination (described in U.S. Pat. Nos. 6,410,266 and 6,361,972, disclosures of which are hereby incorporated by reference in their entireties). This technique allows one to regulate expression to a desired level with a chosen control element while ensuring proper processing and modification of DJ11 endogenously expressed by the host cell. Useful heterologous expression control elements include but are not limited to CMV immediate early promoter, the HSV thymidine kinase promoter, the early and late SV40 promoters, the promoters of retroviral LTRs, such as those of the Rous Sarcoma Virus (RSV), and metallothionein promoters.

Preferably, the expression vector contains a selectable marker gene to allow the selection of transformed host cells. Selection genes are well known in the art and will vary with the host cell used. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available for from complex media, e.g., the gene encoding D-alanine racemase for Bacilli.

Host cells transformed with a nucleotide sequence encoding a DJ11 polypeptide may be cultured under conditions suitable for the expression and recovery of the encoded protein from cell culture. The protein produced by a recombinant cell may be secreted, membrane-bound, or contained intracellularly depending on the sequence and/or the vector used. As will be understood by those of skill in the art, expression vectors containing polynucleotides encoding the DJ11 can be designed with signal sequences which direct secretion of the DJ11 through a prokaryotic or eukaryotic cell membrane. The desired DJ11 polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DJ11-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicas glucoamylase leader (EP 362,179 published Apr. 4, 1990), or the signal described in WO 90113646 published Nov. 15, 1990. In mammalian cell expression, mammalian signal sequences may be used to direct secretion of the protein, such as signal sequences from secreted polypeptides of the same or related species, as well as viral secretory leaders. According to the expression system selected, the coding sequence is inserted into an appropriate vector, which in turn may require the presence of certain characteristic “control elements” or “regulatory sequences.” Appropriate constricts are known generally in the art (Ausubel, et al., 1990) and, in many cases, are available from commercial suppliers such as Invitrogen (San Diego, Calif.), Stratagene (La Jolla, Calif.) Gibco BRL (Rockville, Md.) or Clontech (Palo Alto, Calif.).

Expression in Bacterial Systems.

Transformation of bacterial cells may be achieved using an inducible promoter such as the hybrid lacZ promoter of the “BLUESCRIPT” Phagemid (Stratagene) or “pSPORT1” (Gibco BRL). In addition, a number of expression vectors may be selected for use in bacterial cells to produce cleavable fusion proteins that can be easily detected and/or purified, including, but not limited to “BLUESCRIPT” (a-galactosidase; Stratagene) or pGEX (glutathione S-transferase; Promega, Madison, Wis.). A suitable bacterial promoter is any nucleic acid sequence capable of binding bacterial RNA polymerase and initiating the downstream (3′) transcription of the coding sequence of the DJ11 gene into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tat promoter is a hybrid of the trp and lac promoter sequences. Furthermore, a bacterial promoter can include naturally occurring promoters of non-bacterial origin that have the ability to bind bacterial RNA polymerase and initiate transcription. An efficient ribosome binding site is also desirable. The expression vector may also include a signal peptide sequence that provides for secretion of the DJ11 polypeptide in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include drug resistance genes such as ampicillin, chloramphenicol, erythromycin, kanamycin, neomycin and tetracycline. Selectable markers also include biosynthetic genes, such as those in the histidine, tryptophan and leucine biosynthetic pathways. When large quantities of DJ11 polypeptides are needed, e.g., for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which the DJ11 polypeptide coding sequence may be ligated into the vector in-frame with sequences for the amino-terminal Met and the subsequent 7 residues of beta-galactosidase so that a hybrid protein is produced; PIN vectors [Van Heeke & Schuster JBiol Chem 264:5503-5509 1989)]; PET vectors (Novagen, Madison Wis.); and the like. Expression vectors for bacteria include the various components set forth above, and are well known in the art. Examples include vectors for Bacillus subtilis, E. coli, Sireptococcus cremoris, and Streptococcus lividans, among others. Bacterial expression vectors are transformed into bacterial host cells using techniques well known in the art, such as calcium chloride mediated transfection, electroporation, and others.

Expression in Yeast.

Yeast expression systems are well known in the art, and include expression vectors for Saccharomyces cerevisiae, Candida albicans and C. maltosa, Hansenula polymorpha, Kluyveromyces fragilis and K. lactis, Pichia guillermondii and P pastoris, Schizosaccharomyces pombe, and Yarrowia lipolytica. Examples of suitable promoters for use in yeast hosts include the promoters for 3-phosphoglycerate kinase [Hitzeman et al., J. Biol. Chem. 255:2073 (1980)] or other glycolytic enzymes [Hess et al., J. Adv. Enzyme Reg. 7:149 (1968); Holland, Biochemistry 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, tri-osephosphate isomerase, phosphoglucose isomerase, alpha factor, the ADH2IGAPDH promoter, glucokinase alcohol oxidase, and PGH. [See, for example, Ausubel, et al., 1990; Grant et al., Methods in Enzymology 153:516-544, (1987)]. Other yeast promoters, which are inducible have the additional advantage of transcription controlled by growth conditions, include the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast selectable markers include ADE2. HIS4. LEU2. TRP1. and ALG7, which confers resistance'to tunicamycin, the neomycin phosphotransferase gene, which confers resistance to G418; and the CUP1 gene, which allows yeast to grow in the presence of copper ions. Yeast expression vectors can be constructed for intracellular production or secretion of a DJ11 from the DNA encoding the DJ11 of interest. For example, a selected signal peptide and the appropriate constitutive or inducible promoter may be inserted into suitable restriction sites in the selected plasmid for direct intracellular expression of the DJ11 polypeptide. For secretion of the DJ11, DNA encoding the DJ11 polypeptide can be cloned into the selected plasmid, together with DNA encoding the promoter, the yeast alpha-factor secretory signal/leader sequence, and linker sequences (as needed), for expression of the DJ11 polypeptide. Yeast cells, can then be transformed with the expression plasmids described above, and cultured in an appropriate fermentation media. The protein produced by such transformed yeast can then be concentrated by precipitation with 10% trichloroacetic acid and analyzed following separation by SDS-PAGE and staining of the gels with Coomassie Blue stain. The recombinant DJ11 can subsequently be isolated and purified from the fermentation medium by techniques known to those of skill in the art.

Expression in Mammalian Systems

The DJ11 proteins may be expressed in mammalian cells. Mammalian expression systems are known in the art, and include retroviral vector mediated expression systems. Mammalian host cells may be transformed with any of a number of different viral-based expression systems, such as adenovirus, where the coding region can be ligated into an adenovirus transcription/translation complex consisting of the late promoter and tripartite leader sequence. Insertion in a nonessential E1 or E3 region of the viral genome results in a viable virus capable of expression of the polypeptide of interest in infected host cells. A preferred expression vector system is a retroviral vector system such as is generally described in PCT/US97/01019 and PCT/US97/101048. Suitable mammalian expression vectors contain a mammalian promoter which is any DNA sequence capable of binding mammalian RNA polymerase and initiating the downstream (3′) transcription of a coding sequence for DJ11 protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include promoters obtained from the genomes of viruses such as polyoma virus, fowlpox virus (UK 2,211, 504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems. Transcription of DNA encoding a DJ11 polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer, the cytomegalovirus early promoter enhancer, the polyoma enhancer on the late side of the replication origin, and adenovirus enhancers. The enhancer is preferably located at a site 5′ from the promoter. In general, the transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Long term, high-yield production of recombinant proteins can be effected in a stable expression system. Expression vectors which contain viral origins of replication or endogenous expression elements and a selectable marker gene may be used for this purpose. Appropriate vectors containing selectable markers for use in mammalian cells are readily available commercially and are known to persons skilled in the art. Examples of such selectable markers include, but are not limited to herpes simplex virus thymidine kinase and adenine phosphoribosyltransferase for use in tk- or hprt-cells, respectively. The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

DJ11 polypeptides can be purified from culture supernatants of mammalian cells transiently transfected or stably transformed by an expression vector carrying a DJ11-encoding sequence. Preferably, DJ11 polypeptides are purified from culture supernatants of COS 7 cells transiently transfected by the pcD expression vector. Transfection of COS 7 cells with pcD proceeds as follows: One day prior to transfection, approximately 10⁶ COS 7 monkey cells are seeded onto individual 100 mm plates in Dulbecco's modified Eagle medium (DME) containing 10% fetal calf serum and 2 mM glutamine. To perform the transfection, the medium is aspirated from each plate and replaced with 4 ml of DME containing 50 mM Tris.HCl pH 7.4, 400 mg/ml DEAE-Dextran and 50 μg of plasmid DNA. The plates are incubated for four hours at 37° C., then the DNA-containing medium is removed, and the plates are washed twice with 5 ml of serum-free DME. DME is added back to the plates which are then incubated for an additional 3 hrs at37° C. The plates are washed once with DME, after which DME containing 4% fetal calf serum, 2 mM glutamine, penicillin (100 U/L) and streptomycin (100 μg/L) at standard concentrations is added. The cells are then incubated for 72 hrs at 37° C., after which the growth medium is collected for purification of DJ11 polypeptides. Plasmid DNA for the transfections is obtained by growing pcD(SRα), or like expression vector, containing the DJ11-encoding cDNA insert in E. coli MC1061 (described by Casadaban and Cohen, J. Mol. Biol., Vol. 138, pgs. 179-207 (1980)), or like organism. The plasmid DNA is isolated from the cultures by standard techniques, e.g. Sambrook et al., Molecular Cloning: A Laboratory Manual, Second Edition (Cold Spring Harbor Laboratory, N.Y., 1989) or Ausubel et al (1990, cited above).

Expression in Insect Cells

DJ11 polypeptides may also be produced in insect cells. Expression vectors for the transformation of insect cells, and in particular, baculovirus-based expression vectors, are well known in the art. In one such system, the DJ11-encoding DNA is fused upstream of an epitope tag contained within a baculovirls expression vector. Autographa californica nuclear polyhedrosis virus (AcNPV) is used as a vector to express foreign genes in Spodoptera frugiperda Sf9 cells or in Trichoplusia larvae. The DJ11-encoding sequence is cloned into a nonessential region of the virus, such as the polyhedrin gene, and placed under control of the polyhedrin promoter. Successful insertion of a DJ11-encoding sequence will render the polyhedrin gene inactive and produce recombinant virus lacking coat protein coat. The recombinant viruses are then used to infect S. frugiperda cells or Trichoplusia larvae in which the DJ11 is expressed [Smith et al., J. Wol. 46:584 (1994); Engelhard E K et al., Proc. Nat. Acad. Sci. 91:3224-3227 (1994)]. Suitable epitope tags for fusion to the DJ11-encoding DNA include poly-his tags and immunoglobulin tags (like Fc regions of IgG). A variety of plasmids may be employed, including commercially available plasmids such as pVL1393 (Novagen). Briefly, the DJ11-encoding DNA or the desired portion of the DJ11-encoding DNA is amplified by PCR with primers complementary to the 5′ and 3′ regions. The 5′ primer may incorporate flanking restriction sites. The PCR product is then digested with the selected restriction enzymes and subcloned into an expression vector. Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL), or other methods known to those of skill in the art. Virus is produced by day 4-5 of culture in Sf9 cells at 28° C., and used for further amplifications. Procedures are performed as further described in O'Reilley et al., BACULOVIRUS EXPRESSION VECTORS: A LABORATORY MANUAL, Oxford University Press (1994). Extracts may be prepared from recombinant virus-infected Sf9 cells as described in Rupert et al., Nature 362:175-179 (1993). Alternatively, expressed epitope-tagged DJ11 polypeptides can be purified by affinity chromatography, or for example, purification of an IgG tagged (or Fc tagged) DJ11 polypeptide can be performed using chromatography techniques, including Protein A or protein G column chromatography.

Evaluation of Gene Expression

Gene expression may be evaluated in a sample directly, for example, by standard techniques known to those of skill in the art, e.g., Northern blotting to determine the transcription of mRNA, dot blotting (DNA or RNA), or in situ hybridization, using an appropriately labeled probe, based on the sequences provided herein. Alternatively, antibodies may be used in assays for detection of polypeptides, nucleic acids, such as specific duplexes, including DNA duplexes, RNA duplexes, and DNA-RNA hybrid duplexes or DNA-protein duplexes. Such antibodies may be labeled and the assay carried out where the duplex is bound to a surface, so that upon the formation of duplex on the surface, the presence of antibody bound to the duplex can be detected. Gene expression, alternatively, may be measured by immunohistochemical staining of cells or tissue sections and assay of cell culture or body fluids, to directly evaluate the expression of DJ11 polypeptides or polynucleotides. Antibodies useful for such immunological assays may be either monoclonal or polyclonal, and may be prepared against a native sequence DJ11 based on the DNA sequences provided herein.

Purification of Expressed Protein

Expressed DJ11 polypeptides may be purified or isolated after expression, using any of a variety of methods known to those skilled in the art. The appropriate technique will vary depending upon what other components are present in the sample. Contaminant components that are removed by isolation or purification are materials that would typically interfere with diagnostic or therapeutic uses for the polypeptide, and may include enzymes, hormones, and other solutes. The purification step(s) selected will depend, for example, on the nature of the production process used and the particular DJ11 polypeptide produced. A DJ11 polypeptide or protein may be recovered from culture medium or from host cell lysates. If membrane-bound, it can be released from the membrane using a suitable detergent solution (e.g. Triton-X 100) or by enzymatic cleavage. Alternatively, cells employed in expression of DJ11 polypeptides can be disrupted by various physical or chemical means, such as freeze-thaw cycling, sonication, mechanical disruption, or by use of cell lysing agents. Exemplary purification methods include, but are not limited to, ion-exchange column chromatography; chromatography using silica gel or a cation-exchange resin such as DEAE; gel filtration using, for example, Sephadex G-75; protein A Sepharose columns to remove contaminants such as IgG; chromatography using metal chelating columns to bind epitope-tagged forms of the DJ11 polypeptide; ethanol precipitation; reverse phase HPLC; chromatofocusing; SDS-PAGE; and ammonium sulfate precipitation. Ordinarily, an isolated DJ11 polypeptide will be prepared by at least one purification step. For example, the DJ11 protein may be purified using a standard anti-DJ11 antibody column. Ultrafiltration and dialysis techniques, in conjunction with protein concentration, are also useful (see, for example, Scopes, R., PROTEIN PURIFICATION, Springer-Verlag, New York, N.Y., 1982). The degree of purification necessary will vary depending on the use of the DJ11. In some instances no purification will be necessary. Once expressed and purified as needed, the DJ11 proteins and nucleic acids of the present invention are useful in a number of applications, as detailed below.

Labeling of Expressed Protein

The nucleic acids, proteins and antibodies of the invention may be labeled. By labeled herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position that does not interfere with the biological activity or characteristic of the compound which is being detected.

DJ11 Fusion Proteins

The DJ11 of the present invention may also be modified in a way to form chimeric molecules comprising a DJ11 fused to another, heterologous polypeptide or amino acid sequence. The term “fusion protein” used herein refers to a chimeric polypeptide comprising a DJ11 polypeptide, or domain sequence thereof, fused to a “targeting polypeptide”. The targeting polypeptide has enough residues to facilitate targeting to a particular cell type or receptor, yet is short enough such that it does not interfere with the biological function of the DJ11 polypeptide. The targeting polypeptide preferably is also fairly unique so that the fusion protein does not substantially cross-react with other cell types or receptors. Suitable targeting polypeptides generally have at least about 10 amino acid residues and usually between from about 10 to about 500 amino acid residues. Preferred targeting polypeptides have from about 20 to about 200 amino acid residues. The fusion protein may also comprise a fusion of a DJ11 with a tag polypeptide which provides an epitope to which an anti-tag antibody can selectively bind. The epitope tag is generally placed at the amino-or carboxyl-terminus of the DJ11. Such epitope-tagged forms of a DJ11 can be detected using an antibody against the tag polypeptide. Also, provision of the epitope tag enables the DJ11 to be readily purified by using an anti-tag antibody or another type of affinity matrix that binds to the epitope tag. Alternatively, the fusion protein may comprise a fusion of a DJ11 with an immunoglobulin or a particular region of an immunoglobulin. For a bivalent form of the chimeric molecule, such a fusion could be to the Fc region of an IgG molecule or, for example, GM-CSF. Preferred fusion proteins include, but are not limited to, molecules that facilitate immune targeting of the DJ11. The DJ11 fusion protein may be made for various other purposes using techniques well known in the art. For example, for the creation of antibodies, if the desired epitope is small, a partial or complete DJ11 protein may be fused to a carrier protein to form an immunogen. Alternatively, the DJ11 protein may be made as a fusion protein to increase the ability of the antigen to stimulate cellular and/or humoral (antibody-based) immune responses, or for other reasons.

Synthetic Genes for DJ11s

Once nucleic acid sequence and/or amino acid sequence information is available for a native protein a variety of techniques become available for producing virtually any mutation in the native sequence, e.g.Shortle, in Science, Vol. 229, pgs. 1193-1201 (1985); Zoller and Smith, Methods in Enzymology, Vol. 100, pgs. 468-500 (1983); Mark et al., U.S. Pat. No. 4,518,584; Wells et al., in Gene, Vol. 34, pgs. 315-323 (1985); Estell et al., Science, Vol. 233, pgs. 659-663 (1986); Mullenbach et 20 al., J. Biol. Chem., Vol. 261, pgs. 719-722 (1986), and Feretti et al., Proc. Natl. Acad. Sci., Vol. 83, pgs. 597-603 (1986). Accordingly, these references are incorporated by reference.

Variants of the natural polypeptide (sometime referred to as “muteins”) may be desirable in a variety of circumstances. For example, undesirable side effects might be reduced by certain variants, particularly if the side effect activity is associated with a different part of the polypeptide from that of the desired activity. In some expression systems, the native polypeptide may be susceptible to degradation by proteases. In such cases, selected substitutions and/or deletions of amino acids which change the susceptible sequences can significantly enhance yields, e.g. British patent application 2173-804-A where Arg at position 275 of human tissue plasminogen activator is replaced by Gly or Glu. Variants may also increase yields in purification procedures and/or increase shelf lives of proteins by eliminating amino acids susceptible to oxidation, acylation, alkylation, or other chemical modifications. For example, methionines readily undergo oxidation to form sulfoxides, which in many proteins is associated with loss of biological activity, e.g. Brot and Weissbach, Arch. Biochem. Biophys., Vol. 223, pg. 271 (1983). Often methionines can be replaced by more inert amino acids with little or no loss of biological activity, e.g. Australian patent application AU-A-52451/86. In bacterial expression systems, yields can sometimes be increased by eliminating or replacing conformationally inessential cysteine residues, e.g. Mark et al., U.S. Pat. No. 4,518,584.

Preferably cassette mutagenesis is employed to generate mutant proteins. A synthetic gene is constructed with a sequence of unique (when inserted in an appropriate vector) restriction endonuclease sites spaced approximately uniformly along the gene. The unique restriction sites allow segments of the gene to be conveniently excised and replaced with synthetic oligonucleotides (i.e. “cassettes”) which code for desired mutations. Determination of the number and distribution of unique restriction sites entails the consideration of several factors including (1) preexisting restriction sites in the vector to be employed in expression, (2) whether species or genera-specific codon usage is desired, (3) the number of different non-vector-cutting restriction endonucleases available (and their multiplicities within the synthetic gene), and (4) the convenience and reliability of synthesizing and/or sequencing the segments between the unique restriction sites.

The above technique is a convenient way to effect conservative amino acid substitutions, and the like, in the native protein sequence. “Conservative” as used herein means (i) that the alterations are as conformationally neutral as possible, that is, designed to produce minimal changes in the tertiary structure of the mutant polypeptides as compared to the native protein, and (ii) that the alterations are as antigenically neutral as possible, that is, designed to produce minimal changes in the antigenic determinants of the mutant polypeptides as compared to the native protein. The following is a preferred categorization of amino acids into similarity classes: aromatic (phe, trp, tyr), hydrophobic (leu, ile, val), polar (gln, asn), basic (arg, lys, his), acidic (asp, glu), small (ala, ser, thr, met, gly). Conformational neutrality is desirable for preserving biological activity, and antigenic neutrality is desirable for avoiding the triggering of immunogenic responses in patients or animals treated with the compounds of the invention. While it is difficult to select with absolute certainty which alternatives will be conformationally and antigenically neutral, rules exist which can guide those skilled in the art to make alterations that have high probabilities of being conformationally and antigenically neutral, e.g. Anfisen (cited above); Berzofsky, Science, Vol. 229, pgs. 932-940 (1985); and Bowie et al, Science, Vol. 247, pgs. 1306-1310 (1990). Some of the more important rules include (1) substitution of hydrophobic residues are less likely to produce changes in antigenicity because they are likely to be located in the protein's interior, e.g. Berzofsky (cited above) and Bowie et al (cited above); (2) substitution of physiochemically similar, i.e. synonymous, residues are less likely to produce conformational changes because the replacement amino acid can play the same structural role as the substituted amino acid; and (3) alteration of evolutionarily conserved sequences is likely to produce deleterious conformational effects because evolutionary conservation suggests sequences may be functionally important. In addition to such basic rules for selecting variant sequences, assays are available to confirm the biological activity and conformation of the engineered molecules. Biological assays for the polypeptides of the invention are described more fully above. Changes in conformation can be tested by at least two well known assays: the microcomplement fixation method, e.g. Wasserman et al., J. Immunol., Vol. 87, pgs. 290-295 (1961), or Levine et al. Methods in Enzymology, Vol. 11, pgs. 928-936 (1967) used widely in evolutionary studies of the tertiary structures of proteins; and affinities to sets of conformation-specific monoclonal antibodies, e.g. Lewis et al., Biochemistry, Vol. 22, pgs. 948-954 (1983).

Chemical Manufacture of D11 Compositions

Peptides of the invention are synthesized by standard techniques, e.g. Stewart and Young, Solid Phase Peptide Synthesis, 2nd Ed. (Pierce Chemical Company, Rockford, Ill., 1984). Preferably, a commercial peptide synthesizer is used, e.g. Applied Biosystems, Inc. (Foster City, Calif.) model 430A, and polypeptides of the invention may be assembled from multiple, separately synthesized and purified, peptide in a convergent synthesis approach, e.g. Kent et al, U.S. Pat. No. 6,184,344 and Dawson and Kent, Annu. Rev. Biochem., 69: 923-960 (2000). Peptides of the invention may be assembled by solid phase synthesis on a cross-linked polystyrene support starting from the carboxyl terminal residue and adding amino acids in a stepwise fashion until the entire peptide has been formed. The following references are guides to the chemistry employed during synthesis: Schnolzer et al, Int. J. Peptide Protein Res., 40: 180-193 (1992); Merrifield, J. Amer. Chem. Soc., Vol. 85, pg. 2149 (1963); Kent et al., pg 185, in Peptides 1984, Ragnarsson, Ed. (Almquist and Weksell, Stockholm, 1984); Kent et al., pg. 217 in Peptide Chemistry 84, Izumiya, Ed. (Protein Research Foundation, B. H. Osaka, 1985); Merrifield, Science, Vol. 232, pgs. 341-347 (1986); Kent, Ann. Rev. Biochem., Vol. 57, pgs. 957-989 (1988), and references cited in these latter two references.

Preferably, chemical synthesis of polypeptides of the invention is carried out by the assembly of peptide fragments by native chemical ligation, as described by Dawson et al, Science, 266: 776-779 (1994) and Kent el al, U.S. Pat. No. 6,184,344. Briefly, in the approach, a first peptide fragment is provided with an N-terminal cysteine having an unoxidized sulfhydryl side chain, and a second peptide fragment is provided with a C-terminal thioester. The unoxidized sulfhydryl side chain of the N-terminal cysteine is then condensed with the C-terminal thioester to produce an intermediate peptide fragment which links the first and second peptide fragments with a β-aminothioester bond. The β-aminothioester bond of the intermediate peptide fragment then undergoes an intramolecular rearrangement to produce the peptide fragment product which links the first and second peptide fragments with an amide bond. Preferably, the N-terminal cysteine of internal fragments are protected from undesired cyclization and/or concatenation reactions by a cyclic thiazolidine protecting group as described below. Preferably, such cyclic thiazolidine protecting group is a thioprolinyl group.

Peptide fragments having a C-terminal thioester may be produced as described in the following references, which are incorporated by reference: Kent et al, U.S. Pat. No. 6,184,344; Tam et al, Proc. Natl. Acad. Sci., 92: 12485-12489 (1995); Blake, Int. J. Peptide Protein Res., 17: 273 (1981); Canne et al, Tetrahedron Letters, 36: 1217-1220 (1995); Hackeng et al, Proc. Natl. Acad. Sci., 94: 7845-7850 (1997); or Hackeng et al, Proc. Natl. Acad. Sci., 96: 10068-10073 (1999). Preferably, the method described by Hackeng et al (1999) is employed. Briefly, peptide fragments are synthesized on a solid phase support (described below) typically on a 0.25 mmol scale by using the in situ neutralization/HBTU activation procedure for Boc chemistry dislosed by Schnolzer et al, Int. J. Peptide Protein Res., 40: 180-193 (1992), which reference is incorporated herein by reference. (HBTU is 2-(1H-benzotriazol-1-yl)-1,1,3,3-tetramethyluronium hexafluorophosphate and Boc is tert-butoxycarbonyl). Each synthetic cycle consists of N^{α}-Boc removal by a 1- to 2-minute treatment with neat TFA, a 1-minute DMF flow wash, a 10- to 20-minute coupling time with 1.0 mmol of preactivated Boc-amino acid in the presence of DIEA, and a second DMF flow wash. (TFA is trifluoroacetic acid, DMF is N,N-dimethylformamide, and DIEA is N,N-diisopropylethylamine). N^{α}-Boc-amino acids (1.1 mmol) are preactivated for 3 minutes with 1.0 mmol of HBTU (0.5 M in DMF) in the presence of excess DIEA (3 mmol). After each coupling step, yields are determined by measuring residual free amine with a conventional quantitative ninhydrin assay, e.g. as disclosed in Sarin et al, Anal. Biochem., 117: 147-157 (1981). After coupling of Gln residues, a DCM flow wash is used before and after deprotection by using TFA, to prevent possible high-temperature (TFA/DMF)-catalyzed pyrrolidone formation. After chain assembly is completed, the peptide fragments are deprotected and cleaved from the resin by treatment with anhydrous HF for 1 hour at 0° C. with 4% p-cresol as a scavenger. The imidazole side-chain 2,4-dinitrophenyl (dnp) protecting groups remain on the His residues because the dnp-removal procedure is incompatible with C-terminal thioester groups. However, dnp is gradually removed by thiols during the ligation reaction. After cleavage, peptide fragments are precipitated with ice-cold diethylether, dissolved in aqueous acetonitrile, and lyophilized.

Thioester peptide fragments described above are preferably synthesized on a trityl-associated mercaptopropionic acid-leucine (TAMPAL) resin, made as disclosed by Hackeng et al (1999), or comparable protocol. Briefly, N^{α}-Boc-Leu (4 mmol) is activated with 3.6 mmol of HBTU in the presence of 6 mmol of DIEA and coupled for 16 minutes to 2 mmol of p-methylbenzhydrylamine (MBHA) resin, or the equivalent. Next, 3 mmol of S-trityl mercaptopropionic acid is activated with 2.7 mmol of HBTU in the presence of 6 mmol of DIEA and coupled for 16 minutes to Leu-MBHA resin. The resulting TAMPAL resin can be used as a starting resin for polypeptide-chain assembly after removal of the trityl protecting group with two 1-minute treatments with 3.5% triisopropylsilane and 2.5% H₂O in TFA. The thioester bond can be formed with any desired amino acid by using standard in situ-neutralization peptide coupling protocols for 1 hour, as disclosed in Schnolzer et al (cited above). Treatment of the final peptide fragment with anhydrous HF yields the C-terminal activated mercaptopropionic acid-leucine (MPAL) thioester peptide fragments.

Preferably, thiazolidine-protected thioester peptide fragment intermediates are used in native chemical ligation under conditions as described by Hackeng et al (1999), or like conditions. Briefly, 0.1 M phosphate buffer (pH 8.5) containing 6 M guanidine, 4% (vol/vol) benzylmercaptan, and 4% (vol/vol) thiophenol is added to dry peptides to be ligated, to give a final peptide concentration of 1-3 mM at about pH 7, lowered because of the addition of thiols and TFA from the lyophilized peptide. Preferably, the ligation reaction is performed in a heating block at 37° C. and is periodically vortexed to equilibrate the thiol additives. The reaction may be monitored for degree of completion by MALDI-MS or HPLC and electrospray ionization MS.

After a native chemical ligation reaction is completed or stopped, the N-terminal thiazolidine ring of the product is opened by treatment with a cysteine deprotecting agent, such as O-methylhydroxylamine (0.5 M) at pH 3.5-4.5 for 2 hours at 37 C, after which a 10-fold excess of Tris-(2-carboxyethyl)-phosphine is added to the reaction mixture to completely reduce any oxidizing reaction constituents prior to purification of the product by conventional preparative HPLC. Preferably, fractions containing the ligation product are identified by electrospray MS, are pooled, and lyophilized.

After the synthesis is completed and the final product purified, the final polypeptide product may be refolded by conventional techniques, e.g. Creighton, Meth. Enzymol., 107: 305-329 (1984); White, Meth. Enzymol., 11: 481-484 (1967); Wetlaufer, Meth. Enzymol., 107: 301-304 (1984); and the like. Preferably, a final product is refolded by air oxidation by the following, or like: the reduced lyophilized product is dissolved (at about 0.1 mg/mL) in 1 M guanidine hydrochloride (or like chaotropic agent) with 100 mM Tris, 10 mM methionine, at pH 8.6. After gentle overnight stirring, the re-folded product is isolated by reverse phase HPLC with conventional protocols.

Purification of DJ11 Polypeptides

When polypeptides of the present invention are expressed in soluble form, for example as a secreted product of transformed yeast or mammalian cells, they can be purified according to standard procedures of the art, including steps of ammonium sulfate precipitation, ion exchange chromatography, gel filtration, electrophoresis, or affinity chromatography, e.g. “Enzyme Purification and Related Techniques,” Methods in Enzymology, 22:233-577 (1977), and Scopes, R., Protein Purification: Principles and Practice (Springer-Verlag, New York, 1982). Likewise, when polypeptides of the invention are expressed in insoluble form, for example as aggregates or inclusion bodies, they can be purified by standard procedures in the art, including separating the inclusion bodies from disrupted host cells by centrifugation, solublizing the inclusion bodies with chaotropic and reducing agents, diluting the solubilized mixture, and lowering the concentration of chaotropic agent and reducing agent so that the polypeptide takes on a biologically active conformation. The latter procedures are disclosed in the following references, which are incorporated by reference: Winkler et al, Biochemistry, 25: 4041-4045 (1986); Winkler et al, Biotechnology, 3: 992-998 (1985); Koths et al, U.S. Pat. No. 4,569,790; and European patent applications 86306917.5 and 86306353.3.

DJ11 Polypeptides

The invention provides a pseudogene for a novel Kunitz-type inhibitor, as well as customized or ‘engineered’ active Kunitz-type serine protease inhibitors (i.e., proteins not normally present in nature), methods of making the engineered protease inhibitors, as well as methods of using the engineered protease inhibitors.

The engineered protease inhibitors of the invention are derived from the genomic nucleic acid sequences of a protease inhibitor-like, preferably a Kunitz-type protease inhibitor pseudogene, and have protease inhibition capabilities differing from those of known protease inhibitors. It is thus envisioned that the invention allows the production of protease inhibitors having protease interaction and/or protease inhibition capabilities not present in nature, or enhanced over that present in nature. Altered protease inhibition capabilities include the capability to inhibit a serine protease not substantially inhibited by known protease inhibitors or the capability to inhibit one ore more serine proteases with improved or enhanced efficiency, or both.

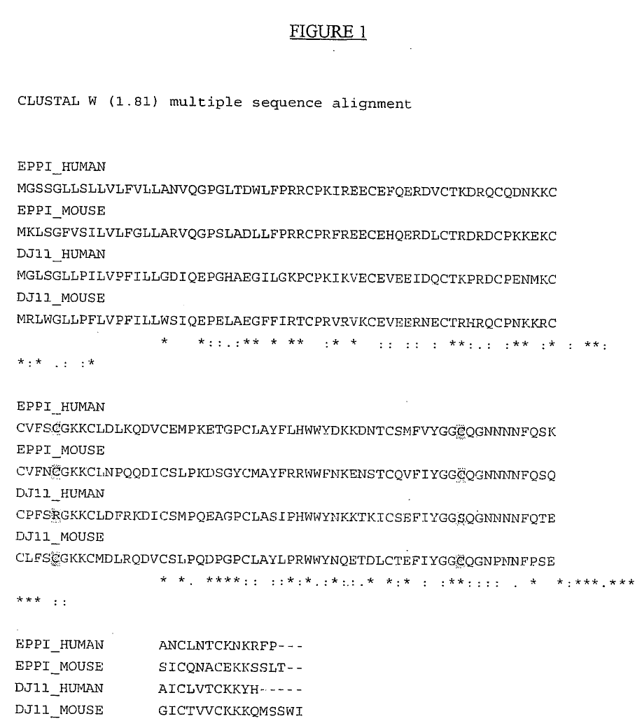

Based on the identification of the human DJ11 nucleic acid sequences described herein, the inventors have designed an active DJ11 protein. Knowledge of the structure and function of the DJ11 pseudogene allows the preparation of a protein capable of inhibiting a serine protease, as well as the preparation or screening of molecules capable of modulating interaction of a biologically active DJ11 protein with a serine protease and/or capable of modulating proteolysis in vitro, in a cell, or in an animal. To design the engineered DJ11 protease inhibitor, the inventors conducted a screen of nucleic acid databases for Kunitz-type protease inhibitor sequences and identified a novel pseudogene having sequence similarity to the gene encoding human Eppin protein (Epididymal protease inhibitor, available from SwissProt at http://www.expasy.ch, under the accession number O95925, and also as SEQ ID NO: 13 herein), the nucleotide sequence of Trembl ref. Q9BQY6, and EST ref. BG216922. Based on the genomic sequence, a cDNA sequence was constructed and modified to delete one adenosine at position 295 (or alternatively any one adjacent adenosine in SEQ ID NO: 8) and add a thymine at position 394 in SEQ ID NO: 11 to form a stop codon (potential stop codon detected in SEQ ID NO: 8), thereby creating a cDNA encoding a polypeptide of 131 amino acid residues. Using Eppin as a scaffold (FIG. 1), one nucleotide was modified such that a cysteine is encoded (CGT→TGT) instead of an arginine at amino acid residue 65, and one nucleotide was modified such that another cysteine is encoded (AGC→TGC) instead of a serine at amino acid residue 110.

Kunitz domains that exist within larger proteins have been shown to retain their functional activities when produced as single domains (Delaria et al., 1997, J. Biol. Chem. 272:12209-12). In one example, a DJ11 polypeptide, or a biologically active fragment or homologue thereof can be the minimum region of a polypeptide that is necessary and sufficient for the inhibition of one or more serine protease of interest. DJ11 activity may be assessed either in vitro (cell or non-cell based) or in vivo depending on the assay type and format. Exemplary assays for protease inhibition activity are further provided herein.

It will be appreciated that the invention comprises polypeptides having an amino acid sequence encoded by any of the polynucleotides of the invention. Provided in SEQ ID NO 2 is a mature form of the engineeered DJ11 polypeptide lacking amino acid residues 1 to 25 of SEQ ID NO 1. The invention thus comprises DJ11 polypeptides with or without a signal sequence directing secretion of the DJ11 polypeptide, as well as polypeptides comprising a biologically active signal peptide of residues 1 to 25 of SEQ ID NO 1 or a fragment thereof.

The present invention embodies isolated, purified, and recombinant polypeptides comprising a contiguous span of at least 6 amino acids, preferably at least 8 to 10 amino acids, more preferably at least 12, 15, 20, 25, 30, 40, 50, or 100 amino acids of SEQ ID NOS 1 or 2. Preferably said contiguous span consists of, consists essentially of, or comprises at least one amino acid residue selected from the group of amino acid positions 1 to 25, 26 to 131 and 77 to 127 of SEQ ID NO 1, and 52 to 102 of SEQ ID NO 2. Also embodied are isolated, purified, and recombinant polypeptides comprising a contiguous span of at least 6 amino acids, preferably at least 8 to 10 amino acids, more preferably at least 12, 15, 20, 25, 30, 40, 50, or 100 amino acids of a polypeptide sequence encoded by a nucleic acid sequence of SEQ ID NO: 11. In other preferred embodiments the contiguous stretch of amino acids comprises the site of a mutation or functional mutation, including a deletion, addition, swap or truncation of the amino acids in the DJ11 protein sequence. The invention also concerns the polypeptide encoded by the DJ11 nucleotide sequences of the invention, or a complementary sequence thereof or a fragment thereof.

It will be appreciated that a functional DJ11 domain may be only a small portion of a DJ11 protein, about amino acids to about 15 amino acids, or from about 20 amino acids to about 25 amino acids, or from about 30 amino acids to about 35 amino acids, or from about 40 amino acids to about 45 amino acids, or from about 50 amino acids to about 55 amino acids, or from about 55 to 60 amino acids. Alternatively, DJ11 activity, as defined above, may require a larger portion of the native protein than may be defined by sequence alignment.

A biologically active DJ11 protein may, for example, comprise at least 1, 2, 3, 5, 10, 20 or 30 amino acid changes from the sequence of SEQ ID NOS 1 or 2 or fragment thereof, or may encode a biologically active DJ11 protein comprising at least 1%, 2%, 3%, 5%, 8%, 10% or 15% changes in amino acids from the sequence of SEQ ID NOS 1 or 2.

Assays

DJ11 proteins, including variants of the engineered DJ11 of SEQ ID NO: 1 or 2, can be tested for activity in protease inhibition assays, a variety of which are known in the art. Assays include, for example, those measuring inhibition of trypsin, chymotrypsin, plasmin, cathepsin G, and human leukocyte elastase which can be carried out as in Petersen et al. (Eur. J. Biochem. 235:310-316, 1996). In a typical procedure, the inhibitory activity of a test compound (e.g., DJ11 polypeptide) is measured by incubating the test compound with the proteinase and adding a substrate of the proteinase, typically a chromogenic peptide substrate. See, for example, Norris et al. (Biol. Chem. Hoppe-Seyler 371:37-42, 1990). Briefly, various concentrations of the inhibitor are incubated in the presence of trypsin, plasmin, and plasma kallikrein in a low-salt buffer at pH 7.4, 25° C. After 30 minutes, the residual enzymatic activity is measured by the addition of a chromogenic substrate (e.g., S2251 (D-Val-Leu-Lys-Nan) or S2302 (D-Pro-Phe-Arg-Nan), available from Kabi, Stockholm, Sweden) and a 30-minute incubation. Inhibition of enzyme activity is indicated by a decrease in absorbance at 405 nm or fluorescence Em at 460 nm. From the results, the apparent inhibition constant Kᵢ is calculated. The inhibition of coagulation factors (e.g., factor VIIa, factor Xa) can be measured using chromogenic substrates or in conventional coagulation assays (e.g., clotting time of normal human plasma).

Inhibition of human leukocyte elastase (HLE) may be assayed as described in U.S. Pat. No. 4,609,667. Briefly, 400 ul of elastase preparation, 10 ul (0.0625 mg) of succinyl-L-alanyl-L-alanyl-L-alanyl-p-nitroanilide (the enzyme substrate), 207 μl of buffer and varying amounts of test elastase inhibitor are incubated at 37 C. for 19 hours. The hydrolysis is measured by use of high pressure liquid chromatography (HPLC) or by the spectrophotometric measurement of the release of p-nitroaniline at a wavelength of 410 nm. Alternatively, the substrate used is an elastin-Congo red complex. Hydrolysis of this substrate by elastase releases Congo red, which is assayed by HPLC. The assay system also contains buffer plus or minus the test elastase inhibitor. The system is incubated at 37 C. for 18 hours. The control sample represents the complete hydrolysis of the elastin-Congo red complex by hog pancreatic elastase. Thus, inhibitory activity is indicated by a reduction in the amount of Congo red released.

DJ11 proteins can also be tested in animal models of disease. Example of animal models include tumor models, models of fibrinolysis, and models of imbalance of hemostasis. Suitable models are known in the art. For example, inhibition of tumor metastasis can be assessed in mice into which cancerous cells or tumor tissue have been introduced by implantation or injection (e.g., Brown, Advan. Enzyme Regul. 35:293-301, 1995; Conway et al., Clin. Exp. Metastasis 14:115-124, 1996). Effects on fibrinolysis can be measured in a rat model wherein the enzyme batroxobin and radiolabeled fibrinogen are administered to test animals. Inhibition of fibrinogen activation by a test compound is seen as a reduction in the circulating level of the label as compared to animals not receiving the test compound. See, Lenfors and Gustafsson, Semin. Thromb. Hemost. 22:335-342, 1996. DJ11 proteins can be delivered to test animals by injection or infusion, or can be produced in vivo by way of, for example, viral or naked DNA delivery systems or transgenic expression.

For example, animal (rabbit) experiments to test inhibition of Human Leukocyte Elastase may be carried out as described in U.S. Pat. No. 5,922,319. Briefly, surgery is preferably performed by a Board-certified ophthalmologist (M.D.). Sedation and euthanasia are performed by a veterinarian (DVM) ophthalmologist (M.D.) under aseptic conditions. Twenty-six rabbits are tranquilized with acetylpromazine maleate, 16 mg/kg intramuscularly. 2% lidocaine HCl eye drops are instilled in each eye for corneal anesthesia. Once the corneas are anesthetized, an eyelid speculum is inserted and the lids are then separated. The corneas of each eye are thermally coagulated with the tip of a battery-powered ophthalmic cautery. A burn measuring 3 mm in diameter is placed near the superior limbus of each eye. Two drops of test inhibitor or control solutions are instilled in designated treated eyes every 15 min for 6 hrs., then every 2 hrs. for 12 hrs. Following this, two eye drops of the same solutions are instilled four times a day. Daily eye examination is performed and photographs are taken. Two weeks after treatment the rabbits are sacrificed by longer intracardiac injection of T-61 (50 ug/kg). Subsequently, the rabbits eyeballs are enucleated, the globes placed in Bounri's solution (10% formalin solution) and submitted for histopathology cell analysis. The eyes are fixed with 10% formalin solution and labeled right (R) and left (L), as appropriate, and sectioned and stained with H & E (hematoxylin & eosin stains). Typical results are obtained from a minimum of six (6) rabbits per treatment. Inhibition of HLE is indicated by a reduction in the severe fibrosis and neovascularization present in the control or untreated corneas.

Anti-DJ11 Antibodies

The present invention provides antibodies and binding compositions specific for DJ11. Such antibodies and binding compositions include polyclonal antibodies, monoclonal antibodies, Fab and single chain Fv fragments thereof, bispecific antibodies, heteroconjugates, and humanized antibodies. Such antibodies and binding compositions may be produced in a variety of ways, including hybridoma cultures, recombinant expression in bacteria or mammalian cell cultures, and recombinant expression in transgenic animals. There is abundant guidance in the literature for selecting a particular production methodology, e.g. Chadd and Chamow, Curr. Opin. Biotechnol., 12: 188-194 (2001).

The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments are highly selective for in vivo tumors, show good tumor penetration and low immunogenicity, and are cleared rapidly from the blood, e.g. Freyre et al, J. Biotechnol., 76: 157-163 (2000). Thus, such molecules are desirable for radioimmununodectection and in situ radiotherapy. Whenever pharmacokinetic activity in the form of increased half-life is required for therapeutic purposes, then full-length antibodies are preferable. For example, the immunoglobulin G (IgG) molecule may be one of four subclasses: γ1, γ2, γ3, or γ4. If a full-length antibody with effector function is required, then IgG subclasses γ1 or γ3 are preferred, and IgG subclass γ1 is most preferred. The γ1 and γ3 subclasses exhibit potent effector function, complement activation, and promote antibody-dependent cell-mediated cytotoxicity through interaction with specific Fc receptors, e.g. Raju et al, Glycobiology, 10: 477-486 (2000); Lund et al, J. Immunol., 147: 2657-2662 (1991).

Polyclonal Antibodies

The anti-DJ11 antibodies of the present invention may be polyclonal antibodies. Such polyclonal antibodies can be produced in a mammal, for example, following one or more injections of an immunizing agent, and preferably, an adjuvant. Typically, the immunizing agent and/or adjuvant will be injected into the mammal by a series of subcutaneous or intraperitoneal injections. The immunizing agent may include DJ11 or a fusion protein thereof. It may be useful to conjugate the antigen to a protein known to be immunogenic in the mammal being immunized. Examples of such immunogenic proteins include, but are not limited to, keyhole limpet hemocyanin (KLH), serum albumin, bovine thyroglobulin, and soybean trypsin inhibitor. Adjuvants include, for example, Freund's complete adjuvant and MPL-TDM adjuvant (monophosphoryl Lipid A, synthetic trehalose dicoryno-mycolate). The immunization protocol may be determined by one skilled in the art based on standard protocols or by routine experimentation.

Monoclonal Antibodies

Alternatively, the anti-DJ11 antibodies may be monoclonal antibodies. Monoclonal antibodies may be produced by hybridomas, wherein a mouse, hamster, or other appropriate host animal, is immunized with an immunizing agent to elicit lymphocytes that produce or are capable of producing antibodies that will specifically bind to the immunizing agent, e.g. Kohler and Milstein, Nature 256:495 (1975). Alternatively, the lymphocytes may be immunized in vitro. The immunizing agent will typically include the DJ11 or a fusion protein thereof. Generally, spleen cells or lymph node cells are used if non-human mammalian sources are desired, or peripheral blood lymphocytes (“PBLs”) are used if cells are of human origin. The lymphocytes are fused with an immortalized cell line using a suitable fusing agent, such as polyethylene glycol, to produce a hybridoma cell, e.g. Goding, MONOCLONAL ANTIBODIES: PRINCIPLES AND PRACTICE, Academic Press, pp. 59-103 (1986); Liddell and Cryer, A Practical Guide to Monoclonal Antibodies (John Wiley & Sons, New York, 1991); Malik and Lillenoj, Editors, Antibody Techniques (Academic Press, New York, 1994). In general, immortalized cell lines are transformed mammalian cells, for example, myeloma cells of rat, mouse, bovine or human origin. The hybridoma cells are cultured in a suitable culture medium that preferably contains one or more substances that inhibit the growth or survival of unfused, immortalized cells. For example, if the parental cells lack the enzyme hypoxanthine guanine phosphoribosyl transferase (HGPRT), the culture medium for the hybridomas typically will include hypoxanthine, aminopterin, and thymidine (HAT), substances which prevent the growth of HGPRT-deficient cells. Preferred immortalized cell lines are those that fuse efficiently, support stable high level production of antibody, and are sensitive to a medium such as HAT medium. More preferred immortalized cell lines are murine or human myeloma lines, which can be obtained, for example, from the American Type Culture Collection (ATCC), Rockville, Md. Human myeloma and mouse-human heteromyeloma cell lines also have been described for the production of human monoclonal antibodies, e.g. Kozbor, J. Immunol. 133:3001 (1984); Brodeur et al., Monoclonal Antibody Production Techniques and Applications, Marcel Dekker, Inc., New York, pp. 51-63 (1987).

The culture medium (supernatant) in which the hybridoma cells are cultured can be assayed for the presence of monoclonal antibodies directed against a DJ11. Preferably, the binding specificity of monoclonal antibodies present in the hybridoma supernatant is determined by immunoprecipitation or by an in vitro binding assay, such as radio-immunoassay (RIA) or enzyme-linked immunoabsorbent assay (ELISA). Appropriate techniques and assays are known in the art. The binding affinity of the monoclonal antibody can, for example, be determined by the Scatchard analysis of Munson and Pollard, Anal. Biochem. 107:220 (1980). After the desired antibody-producing hybridoma cells are identified, the cells may be cloned by limiting dilution procedures and grown by standard methods (Goding, 1986, supra). Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by selected clones may be isolated or purified from the culture medium or ascites fluid by immunoglobulin purification procedures routinely used by those of skill in the art such as, for example, protein A-Sepharose, hydroxyl-apatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be isolated from the DJ11-specific hybridoma cells and sequenced, e.g., by using oligonucleotide probes that are capable of binding specifically to genes encoding the heavy and light chains of murine antibodies. Once isolated, the DNA may be inserted into an expression vector, which is then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for the murine heavy and light chain constant domains for the homologous human sequences (Morrison et al., Proc. Nat. Acad. Sci. 81:6851-6855 (1984); Neuberger et al., Nature 312:604-608 (1984); Takeda et al., Nature 314:452-454 (1985)), or by covalently joining to the immunoglobulin coding sequence all or part of the coding sequence for a non-immunoglobulin polypeptide. The non-immunoglobulin polypeptide can be substituted for the constant domains of an antibody of the invention, or can be substituted for the variable domains of one antigen-combining site of an antibody of the invention to create a chimeric bivalent antibody. The antibodies may also be monovalent antibodies. Methods for preparing monovalent antibodies are well known in the art. For example, in vitro methods are suitable for preparing monovalent antibodies. Digestion of antibodies to produce fragments thereof, particularly, Fab fragments, can be accomplished using routine techniques known in the art.

Antibodies and antibody fragments characteristic of hybridomas of the invention can also be produced by recombinant means by extracting messenger RNA, constructing a cDNA library, and selecting clones which encode segments of the antibody molecule. The following are exemplary references disclosing recombinant techniques for producing antibodies: Wall et al., Nucleic Acids Research, Vol. 5, pgs. 3113-3128 (1978); Zakut et al., Nucleic Acids Research, Vol. 8, pgs. 3591-3601 (1980); Cabilly et al., Proc. Natl. Acad. Sci., Vol. 81, pgs. 3273-3277 (1984); Boss et al., Nucleic Acids Research, Vol. 12, pgs. 3791-3806 (1984); Amster et al., Nucleic Acids Research, Vol. 8, pgs. 2055-2065 (1980); Moore et al., U.S. Pat. No. 4,642,334; Skerra et al, Science, Vol. 240, pgs. 1038-1041(1988); Huse et al, Science, Vol. 246, pgs. 1275-1281 (1989); and U.S. Pat. Nos. 6,054,297; 5,530,101; 4,816,567; 5,750,105; and 5,648,237; which patents are incorporated by reference. In particular, such techniques can be used to produce interspecific monoclonal antibodies, wherein the binding region of one species is combined with non-binding region of the antibody of another species to reduce immunogenicity, e.g. Liu et al., Proc. Natl. Acad. Sci., Vol. 84, pgs. 3439-3443 (1987), and U.S. Pat. Nos. 6,054,297 and 5,530,101. Preferably, recombinantly produced Fab and Fv fragments are expressed in bacterial host systems. Preferably, full-length antibodies are produced by mammalian cell culture techniques. More preferably, full-length antibodies are expressed in Chinese Hamster Ovary (CHO) cells or NSO cells.

Both polyclonal and monoclonal antibodies can be screened by ELISA. As in other solid phase immunoassays, the test is based on the tendency of macromolecules to adsorb nonspecifically to plastic. The irreversibility of this reaction, without loss of immunological activity, allows the formation of antigen-antibody complexes with a simple separation of such complexes from unbound material. To titrate anti-peptide serum, peptide conjugated to a carrier different from that used in immunization is adsorbed to the wells of a 96-well microtiter plate. The adsorbed antigen is then allowed to react in the wells with dilutions of anti-peptide serum. Unbound antibody is washed away, and the remaining antigen-antibody complexes are allowed to react with an antibody specific for the IgG of the immunized animal. This second antibody is conjugated to an enzyme such as alkaline phosphatase. A visible colored reaction produced when the enzyme substrate is added indicates which wells have bound antipeptide antibodies. The use of spectrophotometer readings allows better quantification of the amount of peptide-specific antibody bound. High-titer antisera yield a linear titration curve between 10⁻³ and 10⁻⁵ dilutions.

DJ11 Peptide Antibodies

The invention includes peptides derived from DJ11, and immunogens comprising conjugates between carriers and peptides of the invention. The term immunogen as used herein refers to a substance which is capable of causing an immune response. The term carrier as used herein refers to any substance which when chemically conjugated to a peptide of the invention permits a host organism immunized with the resulting conjugate to generate antibodies specific for the conjugated peptide. Carriers include red blood cells, bacteriophages, proteins, or synthetic particles such as agarose beads. Preferably, carriers are proteins, such as serum albumin, gamma-globulin, keyhole limpet hemocyanin (KLH), thyroglobulin, ovalbumin, or fibrinogen.

The general technique of linking synthetic peptides to a carrier is described in several references, e.g. Walter and Doolittle, “Antibodies Against Synthetic Peptides,” in Setlow et al., eds., Genetic Engineering, Vol. 5, pgs. 61-91 (Plenum Press, N.Y., 1983); Green et al. Cell, Vol. 28, pgs 477-487 (1982); Lerner et al., Proc. Natl. Acad. Sci., Vol. 78, pgs. 3403-3407 (1981); Shimizu et al., U.S. Pat. No. 4,474,754; and Ganfield et al., U.S. Pat. No. 4,311,639. Accordingly, these references are incorporated by reference. Also, techniques employed to link haptens to carriers are essentially the same as the above-referenced techniques, e.g. chapter 20 in Tijssen, Practice and Theory of Enzyme Immunoassays (Elsevier, New York, 1985). The four most commonly used schemes for attaching a peptide to a carrier are (1) glutaraldehyde for amino coupling, e.g. as disclosed by Kagan and Glick, in Jaffe and Behrman, eds. Methods of Hormone Radioimmunoassay, pgs. 328-329 (Academic Press, N.Y., 1979), and Walter et al. Proc. Natl. Acad. Sci., Vol. 77, pgs. 5197-5200 (1980); (2) water-soluble carbodiimides for carboxyl to amino coupling, e.g. as disclosed by Hoare et al., J. Biol. Chem., Vol. 242, pgs. 2447-2453 (1967); (3) bis-diazobenzidine (BDB) for tyrosine to tyrosine sidechain coupling, e.g. as disclosed by Bassiri et al., pgs. 46-47, in Jaffe and Behrman, eds. (cited above), and Walter et al. (cited above); and (4) maleimidobenzoyl-N-hydroxysuccinimide ester (MBS) for coupling cysteine (or other sulfhydryls) to amino groups, e.g. as disclosed by Kitagawa et al., J. Biochem. (Tokyo), Vol. 79, pgs. 233-239 (1976), and Lerner et al. (cited above). A general rule for selecting an appropriate method for coupling a given peptide to a protein carrier can be stated as follows: the group involved in attachment should occur only once in the sequence, preferably at the appropriate end of the segment. For example, BDB should not be used if a tyrosine residue occurs in the main part of a sequence chosen for its potentially antigenic character. Similarly, centrally located lysines rule out the glutaraldehyde method, and the occurrences of aspartic and glutamic acids frequently exclude the carbodiimide approach. On the other hand, suitable residues can be positioned at either end of chosen sequence segment as attachment sites, whether or not they occur in the “native” protein sequence. Internal segments, unlike the amino and carboxy termini, will differ significantly at the “unattached end” from the same sequence as it is found in the native protein where the polypeptide backbone is continuous. The problem can be remedied, to a degree, by acetylating the α-amino group and then attaching the peptide by way of its carboxy terminus. The coupling efficiency to the carrier protein is conveniently measured by using a radioactively labeled peptide, prepared either by using a radioactive amino acid for one step of the synthesis or by labeling the completed peptide by the iodination of a tyrosine residue. The presence of tyrosine in the peptide also allows one to set up a sensitive radioimmune assay, if desirable. Therefore, tyrosine can be introduced as a terminal residue if it is not part of the peptide sequence defined by the native polypeptide.

Peptides can be linked to KLH through cysteines by MBS as disclosed by Liu et al., Biochemistry, Vol. 18, pgs. 690-697 (1979). The peptides are dissolved in phosphate-buffered saline (pH 7.5), 0.1 M sodium borate buffer (pH 9.0) or 1.0 M sodium acetate buffer (pH 4.0). The pH for the dissolution of the peptide is chosen to optimize peptide solubility. The content of free cysteine for soluble peptides is determined by Ellman's method, Ellman, Arch. Biochem. Biophys., Vol.82, pg. 7077 (1959). For each peptide, 4 mg KLH in 0.25 ml of 10 mM sodium phosphate buffer (pH 7.2) is reacted with 0.7 mg MBS (dissolved in dimethyl formamide) and stirred for 30 min at room temperature. The MBS is added dropwise to ensure that the local concentration of formamide is not too high, as KLH is insoluble in >30% formamide. The reaction product, KLH-MBS, is then passed through Sephadex G-25 equilibrated with 50 mM sodium phosphate buffer (pH 6.0) to remove free MBS, KLH recovery from peak fractions of the column eluate (monitored by OD280) is estimated to be approximately 80%. KLH-MBS is then reacted with 5 mg peptide dissolved in 1 ml of the chosen buffer. The pH is adjusted to 7-7.5 and the reaction is stirred for 3 hr at room temperature. Coupling efficiency is monitored with radioactive peptide by dialysis of a sample of the conjugate against phosphate-buffered saline, and may range from 8% to 60%. Once the peptide-carrier conjugate is available, polyclonal or monoclonal antibodies are produced by standard techniques, e.g. as disclosed by Campbell, Monoclonal Antibody Technology (Elsevier, New York, 1984); Hurrell, ed. Monoclonal Hybridoma Antibodies: Techniques and Applications (CRC Press, Boca Raton, Fla., 1982); Schreier et al. Hybridoma Techniques (Cold Spring Harbor Laboratory, N.Y., 1980); U.S. Pat. No. 4,562,003; or the like. In particular, U.S. Pat. No. 4,562,003 is incorporated by reference. The use and generation of fragments of antibodies is also well known, e.g. Fab fragments: Tijssen, Practice and Theory of Enzyme Immunoassays (Elsevier, Amsterdam, 1985); and Fv fragments: Hochman et al. Biochemistry, Vol. 12, pgs. 1130-113.5 (1973), Sharon et al., Biochemistry, Vol. 15, pgs. 1591-1594 (1976) and Ehrlich et al., U.S. Pat. No. 4,355,023; and antibody half molecules: Auditore-Hargreaves, U.S. Pat. No. 4,470,925.

Preferably, monoclonal antibodies, Fv fragments, Fab fragments, or other binding compositions derived from monoclonal antibodies of the invention have a high affinity to DJ11. Preferably such antibodies or compositions bind to the DJ11 polypeptide of SEQ ID NOs: 1 and 2 or fragments thereof with higher affinity than to other polypeptides. The affinity of monoclonal antibodies and related molecules to DJ11 may be measured by conventional techniques including plasmon resonance, ELISA, or equilibrium dialysis. Affinity measurement by plasmon resonance techniques may be carried out, for example, using a BIAcore 2000 instrument (Biacore AB, Uppsala, Sweden) in accordance with the manufacturer's recommended protocol. Preferably, affinity is measured by ELISA, as described in U.S. Pat. No. 6,235,883 for example. Preferably, the dissociation constant between DJ11 and monoclonal antibodies of the invention is less than 10⁻⁵ molar. More preferably, such dissociation constant is less than 10⁻⁸ molar; still more preferably, such dissociation constant is less than 10⁻⁹ molar; and most preferably, such dissociation constant is in the range of 10⁻⁹ to 10⁻¹¹ molar.

Detection of DJ11 Polypeptides

The antibodies of the present invention find use in detection and diagnostic assays for the determination of DJ11, for example, to monitor levels of therapeutically applied DJ11 in an individual. The antibodies of the invention may be used in most assays involving antigen-antibody reactions. The assays may be homogeneous or heterogeneous. In a homogeneous assay approach, the sample can be a biological sample or fluid such as serum, urine, whole blood, lymphatic fluid, plasma, saliva, and the like, cells, tissue, and material secreted by cells or tissues cultured in vitro. The sample can be pretreated if necessary to remove unwanted materials. The immunological reaction usually involves the specific antibody, labeled analyte, and the sample suspected of containing the analyte. The analyte can be directly labeled with the label or indirectly labeled with a means for incorporating the label such as conjugation of the analyte to biotin and having labeled avidin or anti-biotin. The signal from the label is modified, directly or indirectly, upon the binding of the antibody of the labeled analyte. Both the immunological reaction and detection of the extent thereof are carried out in a homogeneous solution. Labels which may be employed as part of a signal producing system capable of producing a signal in relation to the amount of analyte in the sample include free radicals, chromogens, such as fluorescent dyes, chemiluminescers, enzymes, bacteriophages, coenzymes particulate labels and so forth.

In a heterogeneous assay approach, the reagents are usually the sample, the specific antibody, and means for producing a detectable signal. The specimen is generally placed on a support, such as a plate or a slide, and contacted with the antibody in a liquid phase. The support is then separated from the liquid phase and either the support phase or the liquid phase is examined for a detectable signal employing means for producing such signal or signal producing system. The signal is related to the presence of the analyte in the sample. Means for producing a detectable signal includes the use of radioactive labels, fluorescent compounds, enzymes, and so forth. Exemplary of heterogeneous immunoassays are the radioimmunoassay, immunofluorescence methods, and enzyme-linked immunoassays.

One embodiment of an assay employing an antibody of the present invention involves the use of a surface to which the monoclonal antibody of the invention is attached. The underlying structure of the surface may take different forms, have different compositions and may be a mixture of compositions or laminates or combinations thereof. The surface may assume a variety of shapes and forms and may have varied dimensions, depending on the manner of use and measurement. Illustrative surfaces may be pads, beads, discs, or strips which may be flat, concave or convex. Thickness is not critical, generally being from about 0.1 to 2 mm thick and of any convenient diameter or other dimensions. The surface typically will be supported on a rod, tube, capillary, fiber, strip, disc, plate, cuvette and will typically be porous and polyfunctional or capable of being polyfunctionalized so as to permit covalent binding of an antibody and permit bonding of other compounds which form a part of a means for producing a detectable signal. A wide variety of organic and inorganic polymers, both natural and synthetic, and combinations thereof, may be employed as the material for the solid surface. Illustrative polymers include polyethylene, polypropylene, poly(4-methylbutene), polystyrene, polymethracrylate, poly(ethylene terephthalate), rayon, nylon, poly(vinyl butyrate), silicones, polyformaldehyde, cellulose, cellulose acetate, nitrocellulose, and latex. Other surfaces include paper, glasses, ceramics, metals, metaloids, semiconductor materials, cements, silicates, or the like. Also included are substrates that form gels, gelatins, lipopolysaccharides, silicates, agarose and polyacrylamides or polymers which form several aqueous phases such as dextrans, polyalkylene glycols (alkylene of 2 to 3 carbon atoms) or surfactants such as phospholipids. The binding of the antibody to the surface may be accomplished by well known techniques, commonly available in the literature (see, for example, “Immobilized Enzymes,” Ichiro Chibata, Press, New York (1978) and Cuatrecasas, J. Bio. Chem., 245: 3059 (1970)). In carrying out the assay in accordance with this aspect of the invention, the sample is mixed with aqueous medium and the medium is contacted with the surface having an antibody bound thereto. Labels may be included in the aqueous medium, either concurrently or added subsequently so as to provide a detectable signal associated with the surface. The means for producing the detectable signal can involve the use of a second monoclonal antibody having a label conjugated thereto. Separation and washing steps will be carried out as needed. The signal detected is related to the presence of DJ11 in the sample. It is within the scope of the present invention to include a calibration on the same support. A particular embodiment of an assay in accordance with the present invention, by way of illustration and not limitation, involves the use of a support such as a slide or a well of a petri dish. The technique involves fixing the sample to be analyzed on the support with an appropriate fixing material and incubating the sample on the slide with a monoclonal antibody. After washing with an appropriate buffer such as, for example, phosphate buffered saline, the support is contacted with a labeled specific binding partner for the antibody. After incubation as desired, the slide is washed a second time with an aqueous buffer and the determination is made of the binding of the labeled monoclonal antibody to the antigen. If the label is fluorescent, the slide may be covered with a fluorescent antibody mounting fluid on a cover slip and then examined with a fluorescent microscope to determine the extent of binding. On the other hand, the label can be an enzyme conjugated to the monoclonal antibody and the extent of binding can be determined by examining the slide for the presence of enzyme activity, which may be indicated by the formation of a precipitate, color, etc. A particular example of an assay utilizing the present antibodies is a double determinant ELISA assay. A support such as, e.g., a glass or vinyl plate, is coated with an antibody specific for DJ11 by conventional techniques. The support is contacted with the sample suspected of containing DJ11, usually in aqueous medium. After an incubation period from 30 seconds to 12 hours, the support is separated from the medium, washed to remove unbound DJ11 with, for example, water or an aqueous buffered medium, and contacted with an antibody specific for DJ11, again usually in aqueous medium. The antibody is labeled with an enzyme directly or indirectly such as, e.g., horseradish peroxidase or alkaline phosphatase. After incubation, the support is separated from the medium, and washed as above. The enzyme activity of the support or the aqueous medium is determined. This enzyme activity is related to the amount of DJ11 in the sample.

The invention also includes kits, e.g., diagnostic assay kits, for carrying out the methods disclosed above. In one embodiment, the kit comprises in packaged combination (a) a monoclonal antibody more specifically defined above and (b) a conjugate of a specific binding partner for the above monoclonal antibody and a label capable of producing a detectable signal. The reagents may also include ancillary agents such as buffering agents and protein stabilizing agents, e.g., polysaccharides and the like. The kit may further include, where necessary, other members of the signal producing system of which system the label is a member, agents for reducing background interference in a test, control reagents, apparatus for conducting a test, and the like. In another embodiment, the diagnostic kit comprises a conjugate of monoclonal antibody of the invention and a label capable of producing a detectable signal. Ancillary agents as mentioned above may also be present.

Pharmaceutical Compositions

Generally, DJ11 is administered as a pharmaceutical composition comprising an effective amount of DJ11 and a pharmaceutical carrier. A pharmaceutical carrier can be any compatible, non-toxic substance suitable for delivering the compositions of the invention to a patient. Generally, compositions useful for parenteral administration of such drugs are well known, e.g. Remington's Pharmaceutical Science, 15th Ed. (Mack Publishing Company, Easton, Pa. 1980). Alternatively, compositions of the invention may be introduced into a patient's body by implantable or injectable drug delivery system (e.g. Urquhart et al., Ann. Rev. Pharmacol. Toxicol., Vol. 24, pgs. 199-236 (1984); Lewis, ed. Controlled Release of Pesticides and Pharmaceuticals (Plenum Press, New York, 1981); U.S. Pat. No. 3,773,919; U.S. Pat. No. 3,270,960).

Doses of DJ11 proteins will vary according to the severity of the condition being treated and may range from approximately 10 μg/kg to 10 mg/kg body weight, preferably 100 μg/kg to 5 mg/kg, more preferably 100 μg/kg to 1 mg/kg. The proteins are formulated in a pharmaceutically acceptable carrier or vehicle. It is preferred to prepare them in a form suitable for injection or infusion, such as by dilution with sterile water, an isotonic saline or glucose solution, or similar vehicle. In the alternative, the protein may be packaged as a lyophilized powder, optionally in combination with a pre-measured diluent, and resuspended immediately prior to use. Pharmaceutical compositions may further include one or more excipients, preservatives, solubilizers, buffering agents, albumin, to prevent protein loss on vial surfaces, etc. Formulation methods are within the level of ordinary skill in the art. See, Remington: The Science and Practice of Pharmacy, Gennaro, ed., Mack Publishing Co., Easton, Pa., 19th ed., 1995.

Therapeutic Applications of DJ11

The DJ11 proteins are contemplated for use in the treatment or prevention of conditions associated with excessive proteinase activity, such as for example an excess of trypsin, plasmin, kallikrein, thrombin, factor VIIa (TF-FVIIa), TF-FIXa, TF-FXa, TF-FXIa, TF-FXIIa, or matrix metalloproteinases. More preferably, DJ11 proteins are applied to inhibition of neutral serine proteases such as elastase, cathepsin G, proteinase-3. Such conditions include: acute pancreatitis, cardiopulmonary bypass (CPB)-induced pulmonary injury, allergy-induced protease release, deep vein thrombosis, myocardial infarction, shock (including septic shock), hyperfibrinolytic hemorrhage, but especially inflammatory disorders such as emphysema, idiopathic pulmonary fibrosis, adult respiratory distress syndrome, cystic fibrosis, rheumatoid arthritis, glomerulonephritis, chronic inflammatory bowel disease, and psoriasis.

DJ11 proteins are also contemplated for use in preservation of platelet function, organ preservation, and wound healing. DJ11 proteins may also be used within methods for inhibiting blood coagulation in mammals, such as in the treatment of disseminated intravascular coagulation. DJ11 proteins may thus be used in place of known anticoagulants such as heparin, coumarin, and anti-thrombin III. Such methods will generally include administration of the protein in an amount sufficient to produce a clinically significant inhibition of blood coagulation. Such amounts will vary with the nature of the condition to be treated, but can be predicted on the basis of known assays and experimental animal models, and will in general be within the ranges disclosed below. DJ11 proteins may be useful in the treatment of conditions arising from an imbalance in hemostasis, including acquired coagulopathies, primary fibrinolysis and fibrinolysis due to cirrhosis, and complications from high-dose thrombolytic therapy.

The DJ11 proteins of the present invention may be combined with other therapeutic agents to augment the activity (e.g., antithrombotic or anticoagulant activity) of such agents. For example, a DJ11 protein may be used in combination with tissue plasminogen activator in thrombolytic therapy.

### EXAMPLES

### Example 1

### Chemical Synthesis of DJ11

In this example, a DJ11 protein of the invention is synthesized. Peptide fragment intermediates are first synthesized and then assembled into the desired polypeptide.

A DJ11 polypeptide consisting of a DJ11 domain of SEQ ID NO 2 can initally be prepared in 3 fragments, selected to have a Cys residue at the N-terminus of the fragment to be coupled. Fragment 1 is initially coupled to fragment 2 to give a first product, then after preparative HPLC purification, the first product is coupled to fragment 3 to give a second product. Optionally, the DJ11 polypeptide may be produced in 4 or 5 shorter fragments, in which case, after preparative HPLC purification, the second product is coupled to fragment 4 to give a third product. Finally, after preparative HPLC purification, the third product is coupled to fragment 5 to give the desired polypeptide, which is purified and refolded.

Thioester Formation

Fragments 2, 3 and optionally 4 and 5 are synthesized on a thioester generating resin, as described above. For this purpose the following resin is prepared: S-acetylthioglycolic acid pentafluorophenylester is coupled to a Leu-PAM resin under conditions essentially as described by Hackeng et al (1999). In the first case, the resulting resin is used as a starting resin for peptide chain elongation on a 0.2 mmol scale after removal of the acetyl protecting group with a 30 min treatment with 10% mercaptoethanol, 10% piperidine in DMF. The N^{α} of the N-terminal Cys residues of fragments 2 and 3 or 2 through 5 are protected by coupling a Boc-thioproline (Boc-SPr, i.e. Boc-L-thioproline) to the terminus of the respective chains instead of a Cys having conventional N^{α} or S^{β} protection, e.g. Brik et al, J. Org. Chem., 65: 3829-3835 (2000).

Peptide Synthesis

Solid-phase synthesis is performed on a custom-modified 433A peptide synthesizer from Applied Biosystems, using in situ neutralization/2-(1H-benzotriazol-1-yl)-1,1,1,3,3-tetramethyluronium hexafluoro-phosphate (HBTU) activation protocols for stepwise Boc chemistry chain elongation, as described by Schnolzer et al, Int. J. Peptide Protein Res., 40: 180-193 (1992). Each synthetic cycle consists of N^{α}-Boc-removal by a 1 to 2 min treatment with neat TFA, a 1-min DMF flow wash, a 10-min coupling time with 2.0 mmol of preactivated Boc-amino acid in the presence of excess DIEA and a second DMF flow wash. N^{α}-Boc-amino acids (2 mmol) are preactivated for 3 min with 1.8 mmol HBTU (0.5M in DMF) in the presence of excess DIEA (6 mmol). After coupling of Gln residues, a dichloromethane flow wash is used before and after deprotection using TFA, to prevent possible high temperature (TFA/DMF)-catalyzed pyrrolidone carboxylic acid formation. Side-chain protected amino acids are Boc-Arg(p-toluenesulfonyl)-OH, Boc-Asn(xanthyl)-OH, Boc-Asp(O-cyclohexyl)-OH, Boc-Cys(4-methylbenzyl)-OH, Boc-Glu(O-cyclohexyl)-OH, Boc-His(dinitrophenylbenzyl)-OH, Boc-Lys(2-Cl-Z)-OH, Boc-Ser(benzyl)-OH, Boc-Thr(benzyl)-OH, Boc-Trp(formyl)-OH and Boc-Tyr(2-Br-Z)-OH (Orpagen Pharma, Heidelberg, Germany). Other amino acids are used without side chain protection. C-terminal Fragment 1 is synthesized on Boc-Leu-O—CH₂-Pam resin (0.71 mmol/g of loaded resin), while for Fragments 2 through 5 machine-assisted synthesis is started on the Boc-Xaa-S—CH₂—CO-Leu-Pam resin. This resin is obtained by the coupling of S-acetylthioglycolic acid pentafluorophenylester to a Leu-PAM resin under standard conditions. The resulting resin is used as a starting resin for peptide chain elongation on a 0.2 mmol scale after removal of the acetyl protecting group with a 30 min treatment with 10% mercaptoethanol, 10% piperidine in DMF.

After chain assembly is completed, the peptide fragments are deprotected and cleaved from the resin by treatment with anhydrous hydrogen fluoride for 1 hr at 0° C. with 5% p-cresol as a scavenger. In all cases except Fragment 1, the imidazole side chain 2,4-dinitrophenyl (DNP) protecting groups remain on His residues because the DNP-removal procedure is incompatible with C-terminal thioester groups. However DNP is gradually removed by thiols during the ligation reaction, yielding unprotected His. After cleavage, peptide fragments are precipitated with ice-cold diethylether, dissolved in aqueous acetonitrile and lyophilized. The peptide fragments are purified by RP-HPLC with a C18 column from Waters by using linear gradients of buffer B (acetonitile/0.1% trifluoroacetic acid) in buffer A (H₂O/0.1% trifluoroacetic acid) and UV detection at 214 nm. Samples are analyzed by electrospray mass spectrometry (ESMS) using an Esquire instrument (Brücker, Bremen, Germany), or like instrument.

Native Chemical Ligations

As described more fully below, the ligation of unprotected fragments is performed as follows: the dry peptides are dissolved in equimolar amounts in 6M guanidine hydrochloride (GuHCl), 0.2M phosphate, pH 7.5 in order to get a final peptide concentration of 1-8 mM at a pH around 7, and 1% benzylmercaptan, 1% thiophenol is added. Usually, the reaction is carried out overnight and is monitored by HPLC and electrospray mass spectrometry. The ligation product is subsequently treated to remove protecting groups still present. The formyl group of Trp is cleaved by shifting the pH of the solution up to 9.0 with hydrazine and incubating for 1 h at 37° C. Opening of the N-terminal thiazolidine ring further required the addition of solid methoxamine to a 0.5M final concentration at pH3.5 and a further incubation for 2 h at 37° C. A 10-fold excess of Tris(2-carboxyethyl)phosphine is added before preparative HPLC purification. Fractions containing the polypeptide chain are identified by ESMS, pooled and lyophilized.

The ligation of fragments 4 and 5 is performed at pH7.0 in 6 M GuHCl. The concentration of each reactant is 8 mM, and 1% benzylmercaptan and 1% thiophenol were added to create a reducing environment and to facilitate the ligation reaction. An almost quantitative ligation reaction is observed after overnight stirring at 37° C. At this point in the reaction, O—NH₂.HCl is added as a powder to a 0.1 M final concentration and hydrazine is added to shift the pH to 9.0, for the removal of the formyl group of any Trp residues. After 1 h incubation at 37 C., O—NH₂.HCl is further added to the solution to get a 0.5M final concentration, and the pH adjusted to 3.5 in order to open the N-terminal thiazolidine ring. After 2 h incubation at 37° C., ESMS is used to confirm the completion of the reaction. The reaction mixture is subsequently treated with a 10-fold excess of Tris(2-carboxyethylphosphine) over the peptide fragment and after 15 min, the ligation product is purified using the preparative HPLC (e.g., C4, 20-60% CH₃CN, 0.5% per min), lyophilised, and stored at −20 C.

The same procedure is repeated for the remaining ligations with slight modification. Whenever the ligation takes place at an Ile-Cys or Val-Cys site, the ligation reaction is extended to 48 h.

Polypeptide Folding

The full length peptide is refolded by air oxidation by dissolving the reduced lyophilized protein (about 0.1 mg/mL) in 1M GuHCl, 100 mM Tris, 10 mM methionine, pH 8.6 After gentle stirring overnight, the protein solution is purified by RP-HPLC as described above.

### Example 2

### Monoclonal Antibodies Specific for DJ11

A male Lewis rat is immunized with semi-purified preparations of COS 7-cell expressed DJ11. The rat is first immunized with approximately 50 μg of DJ11 in Freund's Complete Adjuvant, and boosted twice with the same amount of material in Freund's Incomplete Adjuvant. Test bleeds are taken. The animal is given a final boost of 25 μg in phosphate-buffered saline, and four days later the spleen is obtained for fusion.

Approximately 3×10⁸ rat splenocytes are fused with an equal number of P3X63-AG8.653 mouse myeloma cells (available from the ATCC under accession number CRL 1580). 3840 microtiter plate wells are seeded at 5.7×10⁴ parental myeloma cells per well. Standard protocols for the fusion and subsequent culturing of hybrids are followed, e.g. as described by Chretien et al, J. Immunol. Meth., Vol. 117, pgs. 67-81 (1989). 12 days after fusion supernatants are harvested and screened by indirect ELISA on PVC plates coated with COS 7-produced DJ11.

### Example 3

### Polyclonal Antibodies Specific for DJ11 Peptide

50 mg of ovalbumin (OVA) and 50 mg of myoglobulin (MYO) (e.g. available from Sigma) are each dissolved in 10 ml of 0.1 M sodium bicarbonate, and reacted with 1 ml of 0.12 iodoacetamide solution (88 mg of iodoacetamide dissolved in 4 ml 0.1 M sodium bicarbonate) for 1 hour at room temperature in a 15 ml Falcon tube (Falcon Plastics, Oxnard, Calif.), or the like. Each reaction mixture is dialyzed overnight against 4 liters of 0.1 M sodium bicarbonate at 4° C. Separately, 10 mg of DJ11 peptide is dissolved in 2 ml of 0.1 M DTT (dithiotheitol) solution (containing 50 mM Tris and 2.5 mM EDTA at pH8) in a 4 ml tube, incubated at 37° C. overnight; and then applied to a GF05 gel-filtration column (1.5×26.5 cm) (LKB, Bromma, Sweden) and eluted with a peptide elution buffer consisting of 0.015 M acetic acid and 0.005 M beta-mercaptoethanol. Three fractions of about 3.5 ml each which contained the reduced peptide are identified by optical density at 206 nm, collected, pooled, frozen in dry ice, and lyophilized overnight. Meanwhile OVA and MYO are recovered from dialysis, and clarified by filtration through 0.45 micrometer filters. OVA and MYO are activated by mixing each with 380 microliters of N-hydroxysuccinimide ester of iodoacetic acid (NHIA) (disclosed by Rector et al., in J. Immunol. Meth., Vol. 24, pg. 321 (1978)) dissolved in tetrahydrofuran (THF) (5 mg/ml); stirring for 30 minutes at room temperature, and dialyzing overnight against 4 liters PBS (1.8 g NaH₂PO₄—H₂O, 7.2 g Na₂HPO₄—H₂O; and 34 g NaCl in 4 liters H₂O). Separately the lyophilized peptide is resuspended in 5 ml of borate reduction buffer (2 g Na₂B₄O₇-10H₂O, 17.4 g NaCl, and 336 mg EDTA-Na₂ in liter H₂O with pH adjusted to 8.5 with concentrated HCl, deoxygenated under nitrogen for 15 minutes, after which 178 mg ascorbate is added). The dialyzed iodoacetylated OVA and MYO are recovered, separately mixed with equal volumes (preferably 2 ml) of borate reduction buffer containing the peptide, and incubated overnight at room temperature. The resulting conjugates are analyzed by SDS-PAGE (12.5% gel). The conjugate containing solution is diluted with PBS to 1 mg/ml, sterile filtered, and aliquotted to convenient volumes (e.g. 500 microliters) for immunizations, and/or stored at 4° C. Polyclonal anti-sera against the MYO conjugate is produced in both rats and rabbits (New Zealand White). The immunization schedule for rabbits is as follows: Initially (week 0) a 10 ml sample of serum is extracted as a control. One week later (week 1) 0.5 ml of peptide-carrier conjugate is mixed with 0.5 ml Freund's Complete Adjuvant and injected I.P. Three weeks later (week 4) a booster is given consisting of 0.5 ml peptide-carrier conjugate mixed with 0.5 ml Freund's Incomplete Adjuvant. The following week (week 5) an additional booster is given, again consisting of 0.5 ml peptide-carrier conjugate mixed with 0.5 ml Freund's Incomplete Adjuvant, followed by yet another identical booster the next week (week 6). On week 7, 20 ml of serum is bled from the animal. After separating out the cellular fraction the serum is assayed for positive anti-DJ11 titer by ELISA. Rat immunization proceed similarly except that the initial injection consists of 0.15 ml PBS and 0.1 ml peptide-carrier conjugate mixed with 0.75 ml Freund's Complete Adjuvant, boosters consisted of 0.15 ml PBS and 0.1 ml peptide-carrier conjugate mixed with 0.75 ml Freund's Incomplete Adjuvant, and only 2-3 ml of serum is bled from the rat. Again, a positive anti-DJ11 reaction is detected by ELISA.

The descriptions of the foregoing embodiments of the invention have been presented for purpose of illustration and description. They are not intended to be exhaustive or to limit the invention to the precise forms disclosed, and obviously many modifications and variations are possible in light of the above teaching. The embodiments were chosen and described in order to best explain the principles of the invention to thereby enable others skilled in the art to best utilize the invention in various embodiments and with various modifications as are suited to the particular use contemplated. It is intended that the scope of the invention be defined by the claims appended hereto. A number of referenced have been cited in the present application. All of the cited references are incoporated herein by reference.

1. An isolated, purified or recombinant DJ11 polypeptide, comprising a Kunitz-type protease inhibitor domain or a biologically active portion thereof, wherein said polypeptide comprises an amino acid sequence having at least 98% amino acid identity to an amino acid sequence selected from the group consisting of:
residues at positions 77 to 127 of SEQ ID NO: 1; and
residues at positions 52 to 102 of SEQ ID NO: 2.

2. The polypeptide of claim 1, wherein said polypeptide or fragment thereof is capable of interacting with a serine protease.

3. The polypeptide of claim 1, wherein said polypeptide or fragment thereof inhibits the catalytic activity of a serine protease.

4. The polypeptide of claim 1, wherein said polypeptide comprises an amino acid sequence at least 95% identical to an amino acid sequence selected from the group consisting of SEQ ID NOS: 1 and 2.

5. The polypeptide of claim 1, wherein said polypeptide comprises an amino acid sequence selected from the group consisting of SEQ ID NOS: 1 and 2.

6. The substantially purified DJ11 polypeptide of claim 1, wherein said polypeptide is encoded by a nucleic acid having the sequence of SEQ ID NO: 11.

7. The isolated, purified or recombinant DJ11 polypeptide according to any one of claims 1 to 5, wherein said polypeptide comprises at least one amino acid deletion, substitution or insertion.

8. A purified or isolated nucleic acid encoding the DJ11 polypeptide according to claim 1, or a biologically active fragment thereof.

9. The isolated or purified nucleic acid encoding a DJ11 polypeptide or a biologically active fragment thereof according to claim 8, wherein said comprising a nucleic acid sequence is at least 90% identical to a nucleotide sequence selected from the group consisting of SEQ ID NOS: 3,8 and 11, and the sequences complementary thereto.

10. The nucleic acid of claim 9, wherein said nucleic acid is operably linked to a promoter.

11. An expression cassette comprising the nucleic acid of claim 10.

12. A host cell comprising the expression cassette of claim 11.

13. A method of making a DJ11 polypeptide, said method comprising
providing a population of host cells comprising a recombinant nucleic acid according to claim 8; and
culturing said population of host cells under conditions conducive to the expression of said recombinant nucleic acid;
whereby said DJ11 polypeptide is produced by said population of host cells.

14. The method of claim 13, further comprising purifying said polypeptide from said population of cells.

15. An isolated DJ11 polypeptide, wherein said polypeptide is encoded by a nucleic acid of SEQ ID NO 11.

16. The isolated DJ11 polypeptide according to claim 1 comprising at least 12 contiguous amino acids of the sequence of SEQ ID NO: 1, wherein said polypeptide possesses at least one DJ11 biological activity.

17. The isolated DJ11 polypeptide or fragment thereof according to claim 1, further comprising at least 8 contiguous amino acids of amino acid residues 1 to 25 of SEQ ID NO: 1.

18. An anti-DJ11 antibody that selectively binds to the polypeptide of any one of claims 1 to 7, or 15 to 17.

19. A method of assessing the biological activity of a DJ11 polypeptide comprising:
(a) providing a DJ11 polypeptide or a fragment thereof; and
(b) assessing the ability of the DJ11 polypeptide to perform a DJ11 biological activity under conditions appropriate for said activity.

20. The method of claim 19, wherein said DJ11 biological activity is inhibition of a protease.

21. A method of determining whether a DJ11 polynucleotide is present in a biological sample, said method comprising the steps of:
(a) contacting a biological sample from a subject with a polynucleotide that hybridizes under stringent conditions to a nucleic acid of claim 8 or 9; and
(b) detecting the presence or absence of hybridization between said polynucleotide and an RNA species within said sample;
wherein a detection of said hybridization indicates that said DJ11 polynucleotide is present in said sample.

22. The method of claim 21, wherein said polynucleotide is a primer, and wherein said hybridization is detected by detecting the presence of an amplification product comprising said primer sequence.

23. A method of determining whether a DJ11 polypeptide is present in a biological sample comprising the steps of:
(a) contacting a biological sample with an anti-DJ11 antibody or DJ11-binding antibody fragment thereof under conditions conducive to antibody binding; and
(b) detecting the binding of said antibody or antibody fragment to a polypeptide in said sample;
wherein a detection of said binding indicates that said DJ11 polypeptide is present in said sample.

24. The isolated or purified nucleic acid according to claim 8, wherein at least a portion of said nucleic acid sequence is at least 90% identical to a nucleotide sequence of nucleotide positions 1 to 75 of SEQ ID NO: 11, and the sequences complementary thereto.

25. The nucleic acid of claim 24, wherein said nucleic acid is operably linked to a nucleic acid encoding a protein of interest.

26. The nucleic acid of claim 25, wherein said protein of interest is a polypeptide according to any one of claims 1 to 7, or 15 to 17.

27. The nucleic acid of claim 8, wherein said nucleic acid comprises the sequence of SEQ ID NO: 11, fragments thereof, and the sequences complementary thereto.

28. The nucleic acid of claim 27, wherein the nucleic acid sequence is degenerate as a result of the genetic code to the sequence of SEQ ID NO: 11.
